(19) United States
(12) Patent Application Publication
(10) Pub. No.: US 2025/0218593 A1
(43) Pub. Date: Jul. 3, 2025

(54) METHODS AND SYSTEMS FOR AN OVERALL HEALTH SCORE

(71) Applicant: ResMed Inc., San Diego, CA (US)

(72) Inventors: Sean COYER, San Mateo, CA (US); Sarah GYATSO, San Diego, CA (US); Jose Ricardo DOS SANTOS, San Diego, CA (US)

(21) Appl. No.: 18/851,846

(22) PCT Filed: Mar. 30, 2023
(86) PCT No.: PCT/US2023/016903

§ 371 Date: Sep. 27, 2024

Related U.S. Application Data
(60) Provisional application No. 63/326,002, filed on Mar. 31, 2022.

(51) Int. Cl. G16H 50/30; G16H 10/60; G16H 50/70

(52) U.S. Cl. CPC G16H 50/30; G16H 10/60; G16H 50/70

(57) ABSTRACT

Techniques are disclosed for monitoring an individual and generating a quantitative score objectively assessing the individual's health. First sensor data, such as from one or more passive sensors in an environment occupied by the individual, can be received over a period of time, such as a number of days. A routine can be identified from this first sensor data. Thereafter, received second sensor data from the same sensor(s) can be leveraged to determine a deviation from the identified routine, which can be used to objectively generate a quantitative score and/or identify context-specific insight(s). The quantitative score and/or context-specific insights can be displayed in a fashion enabling a user to quickly identify changes in the individual's health and how to best manage those changes.

### CROSS-REFERENCE TO RELATED APPLICATIONS

This application claims the benefit of, and priority to, U.S. Provisional Patent Application No. 63/326,002 filed on Mar. 31, 2022, which is hereby incorporated by reference herein in its entirety.

### TECHNICAL FIELD

The present disclosure relates to healthcare generally and more specifically to objectively measuring and presenting an indication of an individual's health, and improving the quality of care of that individual.

### BACKGROUND

Many individuals either are or will be afflicted by certain health conditions in their lifetime. Some health conditions are unknown until a major symptom or incident occurs, while others may grow over time, displaying progressing symptoms. Early detection of health conditions can be important, or even critical, to maintaining the health of the individual. However, it is cost prohibitive and intrusive to constantly be monitoring for many health conditions, especially when no discernable signs indicative of a health condition are present. Further, many types of constant monitoring can be invasive to an individual's privacy.

Such problems are exacerbated when the individual being monitored is unable to self-monitor their actions, when the individual is self-monitoring their but unable or unwilling to share the relevant information with others (e.g., caregivers), and/or when the actions being monitored are not discernable to the naked eye. For example, an individual may be unable to monitor the quality of their own sleep during the night, an individual may be unwilling to share details about the frequency of bathroom visits or frequency of falls with family members, or an individual is progressively taking longer and longer to rise from a chair, but in unnoticeably small increments.

There is a need for techniques to proactively monitor individuals to provide early detection of health conditions. There is a need for techniques to predict future health conditions and provide insight usable for preventative care. There is a need for techniques to present information about an individual's overall health in a fashion that is objective, easy to interpret, and quick to understand.

### SUMMARY

According to some implementations of the present disclosure, a method includes receiving past sensor data from one or more sensors in an environment. The past sensor data is associated with a target individual in the environment. The sensor data is collected over a plurality of past days. The method further includes identifying a routine associated with the target individual based at least in part on the past sensor data. The method further includes receiving current sensor data from the one or more sensors in the environment. The current sensor data is associated with the target individual in the environment. The current sensor data is collected after the plurality of past days. The method further includes determining a deviation from the routine based at least in part on the identified routine and the received current sensor data. The method further includes generating a quantitative score based at least in part on the determined deviation. The method further includes presenting the quantitative score.

In some cases, the method further includes receiving healthcare record information associated with the target individual, wherein generating the quantitative score is further based at least in part on the received healthcare record information. In some cases, generating the quantitative score includes affecting a weighting of the determined deviation based at least in part on the received healthcare record information. In some cases, the method further includes receiving past healthcare record information associated with the target individual, wherein identifying the routine is further based at least in part on the past healthcare record information.

In some cases, the method further includes determining that the deviation is outside of a threshold range. In some cases, the method further includes identifying, based at least in part on at least one of the past sensor data and the current sensor data, one or more context-specific insights associated with the deviation. In some cases, the method further includes presenting an alert in response to determining that the deviation is outside of the threshold range. Presenting the alert includes presenting the one or more context-specific insights. In some cases, determining that the deviation is outside of the threshold range includes determining that the deviation is outside of the threshold range for a threshold duration of time. In some cases, the one or more context-specific insights include i) a duration of time asleep; ii) a duration of time spent in one or more sleep stages; iii) a number of sleep disruptions; iv) a duration of time spent awake after a sleep disruption; v) a room in the environment in which the target individual remains after the sleep disruption; vi) a number of bathroom visits for a given timeframe; vii) a time of bathroom visits; viii) a duration of bathroom visits; ix) a duration of time in bed; x) a duration of time in a sitting position; xi) a start time associated with the duration of time in bed or the duration of time in the sitting position; or xii) any combination of i-xi.

In some cases, presenting the quantitative score further includes presenting a comparison score, wherein the comparison score is a past quantitative score. In some cases, determining the quantitative score includes determining a plurality of component scores based at least in part on the determined deviation, the past sensor data, and the current sensor data; and calculating the quantitative score based on each of the plurality of component scores. In some cases, calculating the quantitative score includes accessing a clinician-supplied weighting for each of the plurality of component scores; and applying, to each of the plurality of component scores, the respective clinician-supplied weighting. In some cases, presenting the quantitative score further includes presenting, for each of the component scores, an indication of an amount the respective component score contributes to the quantitative score. In some cases, presenting the quantitative score includes presenting a comparison score, wherein the comparison score is a past quantitative score, and wherein the comparison score is calculated based on a plurality of past component scores; and presenting, for each of the past component scores, an indication of an amount the respective past component score contributes to the comparison score. In some cases, presenting the indication of the amount the respective component score contributes to the quantitative score and presenting the indication of the amount the respective past component score contributes to the comparison score occur in an overlapping radar plot.

In some cases, the method further includes determining that at least one component score of the plurality of component scores is below a respective threshold score. In some cases, the method further includes selecting a preventative care action in response to determining that the at least one component score is below the respective threshold score, wherein the preventative care action is selected to improve the at least one component score. In some cases, the method further includes facilitating performance of the preventative care action.

In some cases, determining the plurality of component scores includes determining two or more from the group consisting of i) a fall frequency score; ii) an activity score; iii) a sleep score; iv) a bathroom visit score; v) a hygiene score; vi) an infection score; vii) a physical movement score; viii) a mental health score. In some cases, the one or more sensors in the environment include at least one radar sensor. In some cases, the one or more sensors in the environment include at least wearable sensor.

In some cases, the method further includes identifying a change in mental health of the target individual based at least in part on the determined deviation; and generating a mental health component score based at least in part on the identified change in mental health, wherein generating the quantitative score is based at least in part on the metal health component score. In some cases, the determined deviation includes i) a physical deviation from a routine path, ii) a deviation in time spent engaging in self-hygiene tasks, iii) a deviation in time spent engaging with other individuals in the environment, iv) a deviation in time spent engaging in a pre-defined activity, or v) any combination of i-iv.

In some cases, the method further includes generating a physical movement component score based at least in part on the determined deviation, wherein the determined deviation is indicative of i) a change in time to exit a chair; ii) a change in time to sit in a chair; iii) a change in time to cross a room in the environment; iv) a change in time to move from a first point to a second point in the environment; v) a change in gait; or vi) any combination of i-v, wherein generating the quantitative score is based at least in part on the physical movement component score.

In some cases, presenting the quantitative score includes presenting one or more changes in score between the quantitative score and one or more past quantitative scores. In some cases, presenting the quantitative score includes sorting a plurality of quantitative scores associated with a plurality of individuals in the environment, wherein the target individual is one of the plurality of individuals, and wherein the quantitative score is one of the plurality of quantitative scores; and presenting the sorted plurality of quantitative scores.

In some cases, the routine is indicative of i) a pattern of movement of the target individual through the environment; ii) a pattern of sleep of the target individual within the environment; or iii) a combination of i and ii. In some cases, the method further includes determining, based at least in part on the quantitative score and the received past sensor data, that a future quantitative score will drop below a threshold value. In some cases, the method further includes selecting a preventative care action based at least in part on the future quantitative score, wherein the preventative care action is selected to improve the future quantitative score. In some cases, the method further includes facilitating performance of the preventative care action.

Certain aspects and features of the present disclosure relate to a system comprising: a control system including one or more processors; and a memory having stored thereon machine readable instructions; wherein the control system is coupled to the memory, and any one of the methods described above is implemented when the machine executable instructions in the memory are executed by at least one of the one or more processors of the control system.

Certain aspects and features of the present disclosure relate to a system for health scoring for preventative care, the system including a control system configured to implement any one of the methods described above.

Certain aspects and features of the present disclosure relate to a computer program product comprising instructions which, when executed by a computer, cause the computer to carry out any one of the methods described above. In some cases, the computer program product is a non-transitory computer readable medium.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The specification makes reference to the following appended figures, in which use of like reference numerals in different figures is intended to illustrate like or analogous components.

FIG. 1 is a functional block diagram of a system suitable for generating a quantitative health score, according to certain aspects of the present disclosure.

FIG. 2 is an isometric view of an environment which may be occupied by an individual for which a quantitative health score is generated, according to certain aspects of the present disclosure.

FIG. 3 is a flowchart depicting a process for generating a quantitative health score, according to certain aspects of the present disclosure.

FIG. 4 is a schematic diagram depicting a quantitative score and associated component scores, according to certain aspects of the present disclosure.

FIG. 5 is a screenshot of an example graphical user interface for viewing a quantitative health score, according to certain aspects of the present disclosure.

FIG. 6 is a screenshot of an example graphical user interface for comparing current and historical quantitative health scores, according to certain aspects of the present disclosure.

FIG. 7 is a screenshot of an example graphical user interface for viewing an event history associated with a quantitative health score, according to certain aspects of the present disclosure.

FIG. 8 is a screenshot of an example graphical user interface for viewing quantitative health scores for multiple monitored individuals, according to certain aspects of the present disclosure.

FIG. 9 is a screenshot of an example graphical user interface for viewing select event histories for multiple monitored individuals, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Certain aspects and features of the present disclosure relate to techniques for monitoring an individual and generating a quantitative score objectively assessing the individual's health. First sensor data, such as from one or more passive sensors in an environment occupied by the individual, can be received over a period of time, such as a number of days. A routine can be identified from this first sensor data. Thereafter, received second sensor data from the same sensor(s) can be leveraged to determine a deviation from the identified routine, which can be used to objectively generate a quantitative score and/or identify context-specific insight(s).

In an example, an individual may be living in a residential facility with a number of other individuals. Radar sensors installed in the individual's room may be able to track how long it takes for the individual to rise from a seated position and how long it takes for the individual to cross the room from a first point to a second point. After routines have been established for both rising and crossing, deviations from these routines can be identified and used to generate a quantitative health score. For example, if over the course of a period of time it takes the individual longer and longer to rise and cross, the quantitative health score for that individual may decrease accordingly over that period of time. Further, context-specific insights can be identified, such as those specifically related to rising, crossing, or a combination of rising and crossing. In this example, a context-specific insight may be an indication that the longer rise and cross times are indicative of a potential increase in frailty. In some cases, preventative care action can be selected and performance thereof can be facilitated. For example, it can be determined that the individual should engage in additional exercise and balance training, or should receive an additional wellness visit. The system can present a recommendation to take a preventative care action (e.g., display an alert suggesting the individual undergo additional balance training), can automatically take the preventative care action (e.g., automatically order an additional wellness visit), or can otherwise facilitate performance of the preventative care action (e.g., can offer to increase an exercise timer associated with the individual by 10 minutes).

As used herein, the term individual is intended to include anyone who is being monitored by the aspects and features of the present disclosure, such as a resident of an assisted living home or patient in a hospital. As used herein, the term user is intended to include anyone who is reviewing or otherwise interacting with the outputs of the aspects and features of the present disclosure, such as an individual reviewing their own quantitative score, a caregiver monitoring the quantitative score of one or more individuals, or a family member receiving context-specific insights about a remote family member.

Certain aspects and features of the present disclosure can be used in any suitable environment, such as a room, a suite of rooms, a home (e.g., a house, apartment, etc.), an assisted living facility, a hospital, and the like. Other environments can be used. In an example, certain aspects and features of the present disclosure can be used to monitor residents at an assisted living facility or other multi-resident facility. In another example, certain aspects and features of the present disclosure can be used to help an individual monitor themselves at home, or to help a family member monitor another individual at another home (e.g., help someone monitor their elderly parent who lives in another home).

Aspects and features of the present disclosure allow for the monitoring of events (e.g., an individual falling, an individual wandering, an individual visiting the bathroom, an individual falling asleep or waking up, and the like), trends, and other information to identify changes in health status of the individual (e.g., the resident). Users (e.g., the individual being monitored, a caregiver of the individual being monitored, or the like) can then use the changes in health status to decide when is the right time to intervene and provide therapy or take other action (e.g., move the individual to a different facility, provide coaching or therapy, etc.).

One or more sensors can be used to collect sensor data about the individual within the environment. Various sensors can be used, including external sensors (e.g., a radar sensor on a wall of the room), wearable sensors (e.g., a fitness tracker worn by the individual), and internal sensors (e.g., implanted medical devices capable of sharing sensor data). In some cases, sensors can include sensors that leverage active interaction and/or sensors that leverage passive interaction. Active-interaction sensors are those that involve active input from an individual or user, such as a wearable device that needs to be worn or a scale upon which an individual must step to obtain sensor data. Passive-interaction sensors, however, are those that do not require active input from an individual and/or user to collect input, such as a wall-mounted radar sensor that collects data regarding the individual's position within a room without the individual actively interacting with the sensor.

In some cases, the one or more sensors can include one or more sensors capable of indicating an individual's position within the environment or movement through the environment. For example, the sensor can be a camera for monitoring an individual's movement throughout a room. In some cases, the sensor can be a privacy-centric sensor designed to collect positional or movement data without acquiring visual data. For example, the sensor can be a radar sensor designed to receive reflected radio waves (e.g., 300 GHz and below) to identify a location of the individual (e.g., a point cloud indicative of the location of the individual) without obtaining an image of the individual using visual light (e.g., a video image).

Certain aspects and features of the present disclosure relate to determining routines, such as from sensor data. A routine can be a value or set of values (e.g., a number, a coordinate, a time, etc.) associated with an action, activity, or event. In an example, routines associated with an individual going to the bathroom can include an average time spent in the bathroom, an average number of times the individual goes to the bathroom each day, a set of average times or time windows in which the individual most often goes to the bathroom each day, an average number of times the individual goes to the bathroom within a certain limited time window (e.g., between when the individual begins and ends a sleep session), and the like. In another example, routines associated with an individual moving through a room can include an average number of times the individual moves from point A to point B in a day, an average time it takes for the individual to move from point A to point B, an average path the individual follows when moving from point A to point B, and the like. Generally, the value or values associated with a routine can be generated after collecting sensor data for a period of time, such as a number of days (e.g., at least 1, 2, 5, 7, 10, 14, 15, 20, 21, or more days). In some cases, as the system collects additional sensor data throughout a day or throughout a number of days, the routine can be updated accordingly. In some cases, the routine is updated periodically (e.g., once a week), updated on demand (e.g., upon command from a caregiver), or updated dynamically (e.g., automatically as new sensor data is received).

In some cases, a routine can have a preset default value. For example, a preset default value for an individual falling can be zero, such that every instance of the individual falling can be considered a deviation from the routine's (preset default) value.

Certain aspects and features of the present disclosure relate to determining deviations from a routine. A deviation from a routine can be one or more values that differ from the one or more values of the routine by a threshold amount. The threshold amount can be preset (e.g., a deviation of at least 1 unit or more, a deviation of at least 0.8 standard deviations or more, etc.), can be user-selectable (e.g., the user makes a selection indicative of a particular number of units or a particular z-score), or can be dynamically adjusted (e.g., the number of units or z-score can automatically change based on the value(s) associated with the routine, based on the value(s) associated with another routine, or based on other information associated with the individual, such as the individual's healthcare information). In some cases, a routine can represent an average value or values associated with an activity (e.g., an average time spent in the bathroom or an average path taken when crossing a room). In such cases, deviations from the routine can be based on the z-score, or the number of standard deviations away from the average. For example, for certain activities, a deviation may occur when the current value associated with the activity is greater than one standard deviation away from the routine (e.g., a z-score below −1 or greater than 1).

In some cases, determining a deviation from a routine can include detecting that a current value or values associated with a particular activity exceeds the value or values associated with the routine by a threshold amount. For example, with respect to falling, a deviation from a routine can be whenever the number of times the individual falls is greater than one.

In some cases, however, determining a deviation from a routine can include detecting a trend in the current value or values associated with a particular activity exceeding the value or values associated with the routine by threshold amounts. For example, with respect to rising from a seated position, a deviation may be determined when the value or values associated with the routine have increased by a threshold amount over a threshold duration of time (e.g., the average time to rise from a seated position has increased from 4 seconds to 8 seconds over the course of a week).

Certain aspects of the present disclosure are able to take into account information from one or both of the individual's activities of daily living (ADL) and electronic healthcare records (EHR). Thus, the quantitative score and/or context-specific insights can take into account information from both ADL and EHR sources.

ADL information can be obtained from the sensor data, and can include information about the individual's self-care activities, such as bathing and showering (e.g., frequency or time spent bathing and showering), personal hygiene and grooming (e.g., frequency or time spent combing hair, brushing teeth, shaving, etc.), dressing (e.g., frequency or time spent dressing, timing of when dressing occurs), toilet hygiene (e.g., frequency or time spent going to the bathroom), functional mobility (e.g., time to get in or out of bed, time to get in or out of a chair or other seat, time to walk from a first point to a second point, etc.), and self-feeding (e.g., frequency or time spent feeding oneself). Further, ADL information can include information about an individual's reliance on others or assistive devices for any self-care activities (e.g., how often a caregiver must assist the individual in feeding themselves or how often the individual uses a walking aid to walk from a first point to a second point in the environment).

EHR, also referred to as healthcare records, can be obtained via manual input and/or from a network-accessible server or other network connection. Healthcare records include information about the individual's health or care from other sources, such as information about medications (e.g., records of prescribed medications or records of administration of medication), procedures (e.g., records of medical procedures or interventions), caregiver visits (e.g., visits by doctors or other healthcare professionals), and the like. In some cases, healthcare records can include additional health-related or health-tangential information, such as visitor data (e.g., information about family member visits), entertainment activity data (e.g., information about entertainment activities for which the individual has signed or in which the individual has participated), personal preference data (e.g., information about the individual's personal preferences, such as food preferences), and the like.

In an example, the system can monitor an individual's movements throughout the environment to identify when the individual enters the bathroom and when the individual exits the bathroom. The system can then determine an average time the individual spends in the bathroom, which can be a bathroom routine. If the system detects that the average time the individual spends in the bathroom has increased significantly in the past several days, or detects that the average time the individual spends in the bathroom is increasing each day over the past several days, the system may determine that a deviation has occurred. The system may then indicate a lower quantitative score as a result of the deviation and/or may provide a context-specific insight indicative that the individual may be suffering from a urinary tract infection. However, before indicating the lower quantitative score and/or providing the context-specific insight, the system may determine, from the EHR, that the individual has recently began a course of diuretic medication, and thus conclude that the increased time in the bathroom is expected, and thus not indicate the lower quantitative score and/or not provide the context-specific insight.

Certain aspects and features of the present disclosure relate to techniques for presenting health information about individual(s) in an informative and easy-to-understand fashion. The use of a quantitative score permits a user to quickly assess how the individual is faring, especially when compared with other individuals. For example, in a healthcare facility with multiple residents, a caregiver monitoring the residents may be able to access a dashboard displaying the quantitative score of each resident, thus quickly being able to ascertain which resident(s) may need additional assistance and/or which residents are improving or declining. Further, the use of a quantitative score permits a user to readily compare the individual's health on any given day to the individual's health on previous days, thus providing an indication of whether the individual's health is improving or declining.

The quantitative score, also known as a total health score, can be generated through analysis of the sensor data (e.g., analysis of the sensor data or its outputs, such as detected deviations from routines) and/or EHR data. In some cases, the quantitative score can be generated through the combination of multiple component scores, each of which can be generated through analysis of the sensor data and/or EHR data.

In an example, a total health score can be generated from a combination of component scores including at least two of a mobility score, a sleep score, a social score, a physical score, a respiratory/cardiovascular score, and a mental health score. Each of these component scores can be generated based at least in part on sensor data and/or EHR data. In some cases, generating the total health score can include applying a respective weighting to each component score. Such weightings can be preset, user-set, set by a healthcare provider, dynamically adjusted (e.g., an individual having difficulty sleeping or whose sleep fluctuates often may have their sleep score weighted more strongly than an individual who regularly sleeps well), or otherwise set.

Certain aspects and features of the present disclosure relate to a graphical user interface (GUI) that presents a dashboard for viewing the total health score and optionally one or more component scores and/or one or more detected deviations. In an example, a dashboard may display the individual's name, a photograph of the individual, additional identifying information or demographic information about the individual (e.g., the individual's age and room number), a current total health score, a selection of some or all of the component scores, and a listing of detected deviations for an activity (e.g., listing of detected falls within the past three months).

In some cases, presenting one or more components scores can include presenting numerical values for the component scores. In some cases, presenting one or more components cores can include generating and presenting a radar plot having each component score on a separate radial. In some cases, the range of each radial can be normalized (e.g., each component score represented as a value out of 100) or can be based on an amount of contribution that score provides to the total health score (e.g., a component score that accounts for 20% of the total health score may have a radial of a particular length that represents a range from a minimum of 0 to a maximum of 20, while a component score that accounts for 15% of the total health score may have a radial of the same length that represents a range from a minimum of 0 to a maximum of 15). By plotting each component score on its own radial, a shape can be generated by using each plotted point as a vertex, which shape can help indicate how the individual is performing across the various component scores and/or how much each component score is affecting the total health score.

In some cases, a quantitative score and/or a component score can be present at the same time as a historical quantitative score and/or historical component score, respectively. A historical score can be a score that occurred in the past, such as an immediately prior score, a score from a certain number of minutes ago (e.g., a score from two hours ago), a score from a certain number of days ago (e.g., yesterday's score), and the like (e.g., last month's score, last year's score, etc.). In some cases, the historical score can be associated with a change in circumstance or an event. For example, a historical score can be a score from before the most recent falling episode, in which case the comparison between the current score and the historical score can be comparing the score from after the individual has fallen with the score from before the individual fell. In some cases, multiple component scores can be presented at the same time as their respective historical component scores, such as using the radar plot described herein. For example, the shape defined by the current component scores can be overlaid on top of the shape defined by the historical component scores, in which case a user can easily picture how the particular component scores have changed with respect to one another. In some cases, presenting a current quantitative score and a historical quantitative score can include presenting each score as a ring (e.g., a circular ring or ring of another suitable shape) filled according to each score's percentage out of 100%. The current rings can be concentric, with the current quantitative score being indicated in a larger ring having a diameter larger than the ring indicating the historical quantitative score. Thus, a user will be able to easily and quickly see and understand the current quantitative score, as well as how it has changed with respect to the historical quantitative score. Other techniques for displaying score(s) can be used.

Certain aspects of the present disclosure can be used to generate context-specific insights. The context-specific insights can be generated based at least in part on the sensor data, healthcare record information, determined routines, deviations from routines, quantitative scores, component score(s), or any combination thereof. The context-specific insights can be provided in response to determining that a deviation from a routine has exceeded a threshold value, in response to a quantitative score dropping below a threshold minimum, in response to a component score dropping below a threshold minimum, or any combination thereof.

In some cases, the context-specific insight can be an indication of a deviation from a routine. For example, if the system identifies a deviation that the individual has used the bathroom more in the past day than provided by the determined routine, the context-specific insight may be an alert indicating that the individual “has visited the bathroom more than usual in the past 24 hours.”

In some cases, the context-specific insight can be an indication of a change in a quantitative score or component score. For example, if the system identifies that an individual's sleep score has increased in the few days with respect to a previous sleep score established throughout the week prior, the context-specific insight may be an alert indicating that the individual “has been sleeping better in the past few days than they have been in the past week.”

In some cases, a context-specific insight can be an insight designed to facilitate diagnosing the individual with a health condition or ascertaining progression of a health condition. In an example, if the system identifies that the individual is taking longer to rise from a seated position and deviating more from routine paths through the environment, and the system accesses healthcare information indicative that the individual has been diagnosed with Alzheimer's disease, the context-specific insight may be an alert indicating that the individual “appears to be having more difficulty with standing and walking, which may indicate progression of their Alzheimer's disease diagnosis.”

In some cases, in addition to or instead of a context-specific insight, the system can select a preventative care action and facilitate performance of the preventative care action. Selecting the preventative care action can occur similarly to identifying a context-specific insight. The preventative care action can be selected based at least in part on the sensor data, healthcare record information, determined routines, deviations from routines, quantitative scores, component score(s), or any combination thereof. The preventative care action can be selected in response to determining that a deviation from a routine has exceeded a threshold value, in response to a quantitative score dropping below a threshold minimum, in response to a component score dropping below a threshold minimum, or any combination thereof. Facilitating performance of the preventative care action can include presenting an alert indicating the preventative care action to be taken, presenting an alert indicating actions to take to perform the preventative care action, automatically instigating performance of the preventative care action (optionally after receiving user or professional confirmation), or any combination thereof.

In an example, if the system identifies that the individual's mobility component score is decreasing, a preventative care selection can be made that the individual should engage in additional physical therapy. This preventative care action (e.g., additional physical therapy) can then be facilitated, such as by (i) presenting an alert to a caregiver indicating that the individual “appears to be having more difficulty moving around, and may benefit from additional physical therapy; (ii) presenting an alert to the individual indicating that “physical therapy may be beneficial” and presenting instructions for engaging in the desired physical therapy exercises; (iii) automatically scheduling a physical therapy session with a physical therapist for some time in the future; (iv) automatically requesting a caregiver (e.g., a clinician) reach out to the individual to assess the individual to see if physical therapy is warranted; or (v) any combination of i-iv.

Certain aspects and features of the present disclosure greatly improve the ability to objectively assess an individual's health over a period of time, such as a period of days, months, or even years. Certain aspects and features of the present disclosure are able to keep track of and analyze more information than would be possible by a human, and can discern information that would be otherwise unnoticeable to the human eye (e.g., slight variations in gait or deviations from routine routes through a room) or infeasible for a human to monitor (e.g., monitoring the time it takes for an individual to rise from a seated position every time the individual rises from a seated position, rather than only during specific monitoring sessions).

Further, certain aspects and features of the present disclosure greatly improve the ability for a user to review and keep track of health information for a single individual or multiple individuals. Various GUI improvements allow a user to rapidly identify individuals with falling quantitative scores, and allows the user to rapidly identify what component scores are attributing to that individual's falling quantitative score. Further, the identification and presentation of context-specific insights allow a user to quickly and accurately identify the reasoning behind detected deviations and/or changing score(s).

Since it is impractical and infeasible for caregivers to constantly monitor an individual, much less multiple individuals, certain aspects and features of the present disclosure enable benefits that are unavailable when individuals are being monitored by humans, rather than by the systems and methods as disclosed herein.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

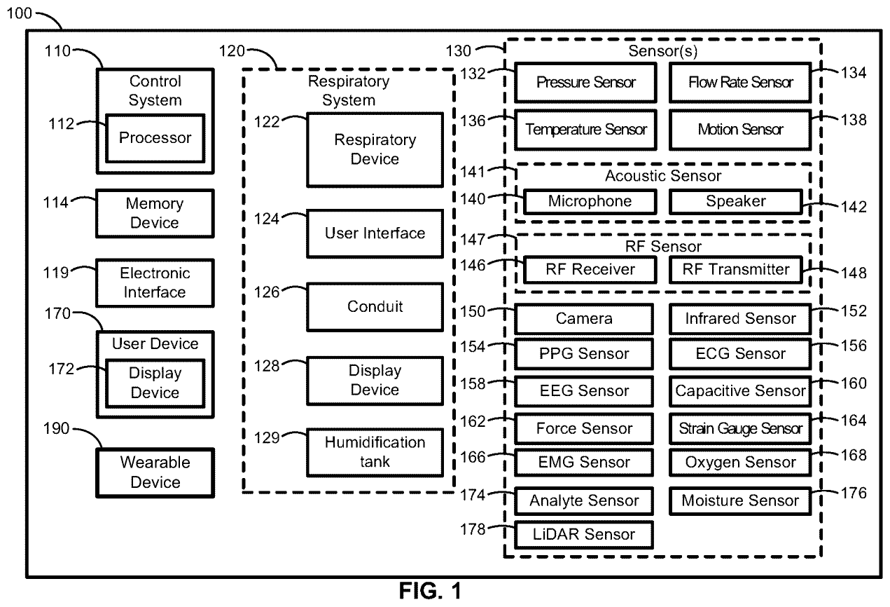

FIG. 1 is a functional block diagram of a system 100 suitable for generating a quantitative health score, according to certain aspects of the present disclosure. The system 100 includes a control system 110, a memory device 114, and one or more sensors 130. In some cases, system 100 can optionally include an electronic interface 119, one or more user devices 170, one or more wearable devices 190, and/or a respiratory therapy system 120.

In some cases, a single system 100 can be used to monitor a single individual or multiple individuals. In some cases, the system 100 can use multiple sets of one or more sensors 130, with each set of one or more sensors being associated with and used to monitor a different individual. In some cases, however, a single set of one or more sensors 130 can be used to monitor either a single individual or multiple individuals. In some cases, multiple iterations of system 100 can be used to monitor multiple individuals.

The control system 110 includes one or more processors 112 (hereinafter, processor 112). The control system 110 is generally used to control the various components of the system 100 and/or analyze data obtained and/or generated by the components of the system 100. The processor 112 can be a general or special purpose processor or microprocessor. While one processor 112 is depicted, the control system 110 can include any suitable number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of a user device 170, a housing of a wearable device 190, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is depicted, the system 100 can include any suitable number of memory devices 114 (e.g., one memory device, two memory devices, five memory devices, ten memory devices, etc.). The memory device 114 can be coupled to and/or positioned within the same housing as the control system 110, or another housing. Like the control system 110, the memory device 114 can be centralized (within one such housing) or decentralized (within two or more of such housings, which are physically distinct).

In some implementations, the memory device 114 stores sensor data (e.g., current and/or historical data from one or more sensors), routine data (e.g., data about one or more current and/or historical routines), deviation data (e.g., data about one or more current and/or historical deviations from routines), score data (e.g., data about one or more current and/or historical scores, including quantitative scores and/or component scores), context-specific insight data (e.g., data about one or more current and/or historically provided context-specific insights, and/or data about how to identify context-specific insights), preventative care action data (e.g., data about one or more current and/or historical preventative care actions, and/or data about how to select preventative care actions), individual identification/demographic information (e.g., identification and/or demographic information about an individual), healthcare data (e.g., healthcare information associated with the individual), customization data (e.g., data regarding individual-specific customizations, such as customized weighting factors for generating quantitative scores from component scores), or any combination thereof.

In some cases, healthcare data includes fall risk assessment data associated with the individual (e.g., a fall risk score using the Morse fall scale), a multiple sleep latency test (MSLT) test result or score, and/or a Pittsburgh Sleep Quality Index (PSQI) score or value.

In some cases, the system 100 includes an electronic interface 119, which can be configured to receive data from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in a user device 170 or a sensor of the one or more sensors 130. In some cases, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory pressure therapy device 122 (referred to herein as respiratory device 122), a user interface 124 (also referred to as a mask or a patient interface), a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a therapy user's airways at a controlled target pressure that is nominally positive with respect to atmosphere throughout the therapy user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air (e.g., using one or more motors (such as a blower motor) that drive one or more compressors) that is delivered to an individual making use of respiratory therapy, which can be known as a therapy user. As used herein, a therapy user (e.g., individual making use of respiratory therapy) can be distinct from a user of system 100 (e.g., someone, such as a caregiver, using system 100 to monitor the individual making use of respiratory therapy). In some implementations, the respiratory device 122 generates continuous constant air pressure that is delivered to the therapy user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the therapy user's face and delivers pressurized air from the respiratory device 122 to the therapy user's airway to aid in preventing the airway from narrowing and/or collapsing during sleep. This may also increase the therapy user's oxygen intake during sleep. Depending upon the therapy to be applied, the user interface 124 may form a seal, for example, with a region or portion of the therapy user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

In some implementations, the user interface 124 is a face mask that covers the nose and mouth of the therapy user. Alternatively, the user interface 124 can be a nasal mask that provides air to the nose of the therapy user or a nasal pillow mask that delivers air directly to the nostrils of the therapy user. The user interface 124 can include a plurality of straps (e.g., including hook and loop fasteners) for positioning and/or stabilizing the interface on a portion of the therapy user (e.g., the face) and a conformal cushion (e.g., silicone, plastic, foam, etc.) that aids in providing an air-tight seal between the user interface 124 and the therapy user. In some examples, the user interface 124 can be a tube-up mask, wherein straps of the mask are configured to act as conduit(s) to deliver pressurized air to the face or nasal mask. The user interface 124 can also include one or more vents for permitting the escape of carbon dioxide and other gases exhaled by the therapy user 210. In other implementations, the user interface 124 can comprise a mouthpiece (e.g., a night guard mouthpiece molded to conform to the therapy user's teeth, a mandibular repositioning device, etc.).

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user's airway. Thus, the air pathway generally includes at least a motor of the respiratory therapy device 122, the user interface 124, and the conduit 126.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., sleep performance metrics, a sleep performance score, a sleep score, a therapy-specific score (such as a myAir™ score, such as described in WO 2016/061629 and US 2017/0311879, each of which is hereby incorporated by reference herein in its entirety), a total health score as disclosed in further detail herein, one or more component scores as disclosed in further detail herein, the current date/time, personal information for the therapy user, questionnaire for the user, etc.). In some implementations, the display device 128 acts as a human-machine interface (HMI) that includes a GUI configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human individual interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the therapy user. Additionally, in some implementations, the conduit 126 can also include a heating element (e.g., coupled to and/or imbedded in the conduit 126) that heats the pressurized air delivered to the therapy user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

The respiratory system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the therapy user. The APAP system automatically varies the air pressure delivered to the therapy user based on, for example, respiration data associated with the therapy user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory device 122. In some cases, the respiratory system 120, such as one or more sensor of the respiratory system 120, can be used to obtain sensor data used to determine routines, identify deviations from routines, or otherwise inform generation of quantitative scores and/or component scores. In an example, a respiratory/cardiovascular component score may be based at least in part on sensor data from one or more sensors of a respiratory system 120.

The user interface 124 (e.g., a full face mask) can be worn by a therapy user during the therapy user's sleep session. The user interface 124 is fluidly coupled and/or connected to the respiratory device 122 via the conduit 126. In turn, the respiratory device 122 delivers pressurized air to the therapy user via the conduit 126 and the user interface 124 to increase the air pressure in the throat of the therapy user to aid in preventing the airway from closing and/or narrowing during sleep. The respiratory therapy device 122 can include the display device 128, which can allow the user to interact with the respiratory therapy device 122. The respiratory therapy device 122 can also include the humidification tank 129, which stores the water used to humidify the pressurized air. The respiratory therapy device 122 can be positioned on a nightstand that is directly adjacent to the therapy user's bed, or more generally, on any surface or structure that is generally adjacent to the bed and/or the therapy user. The therapy user can also wear, for example, a blood pressure device and/or wearable device 190 while lying on the mattress in the bed.

System 100 can include one or more sensors 130. These one or more sensors 130 can include a pressure sensor 132, a flow rate sensor 134, temperature sensor 136, a motion sensor 138, a microphone 140, a speaker 142, a radio-frequency (RF) receiver 146, an RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate sensor data, such as movement data, position data, physiological data, audio data, or the like. Movement data can include data indicative of an individual's movement through an environment. Position data can include data indicative of an individual's position within the environment. In an example, movement data and position data can be a set of point clouds captured over time each indicating the individual's position at that point in time, which, when combined, provides an indication of the individual's movements during that time.

Physiological data generated by one or more of the sensors 130 can be used by the control system 110 to determine a sleep-wake signal associated with an individual during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. N1 and N2 can be considered light sleep stages, whereas N3 can be considered a deep sleep stage. Methods for determining sleep stages from physiological data generated by one or more of the sensors, such as sensors 130, are described in, for example, WO 2014/047310, U.S. Pat. Nos. 10,492,720, 10,660,563, US 2020/0337634, WO 2017/132726, WO 2019/122413, US 2021/0150873, WO 2019/122414, US 2020/0383580, each of which is hereby incorporated by reference herein in its entirety. The sleep-wake signal can also be timestamped to indicate a time that the individual enters the bed (e.g., as determined by movement and/or position data), a time that the individual exits the bed (e.g., as determined by movement and/or position data), a time that the individual attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the individual during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with an individual during a sleep session. The respiration signal is generally indicative of respiration or breathing of the individual during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, RERAs, a flow limitation (e.g., an event that results in the absence of the increase in flow despite an elevation in negative intrathoracic pressure indicating increased effort), a mask leak (e.g., from the user interface 124), a restless leg, a sleeping disorder, choking, an increased heart rate, labored breathing, an asthma attack, an epileptic episode, a seizure, increased blood pressure, hyperventilation, or any combination thereof. Events can be detected by any means known in the art such as described in, for example, U.S. Pat. Nos. 5,245,995, 6,502,572, WO 2018/050913, WO 2020/104465, each of which is incorporated by reference herein in its entirety.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the therapy user using the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof. In one example, the pressure sensor 132 can be used to determine a blood pressure of an individual.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of an individual, a skin temperature of an individual, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the individual and/or detect movement of any components of system 100. The motion sensor 138 can include one or more inertial sensors, such as accelerometers, gyroscopes, and magnetometers. The motion sensor 138 can be used to detect motion or acceleration associated with arterial pulses, such as pulses in or around the face of the individual and proximal to the user interface 124, and configured to detect features of the pulse shape, speed, amplitude, or volume. In some implementations, the motion sensor 138 alternatively or additionally generates one or more signals representing bodily movement of the individual, from which may be obtained movement data and/or position data. In some cases, one or more signals representing bodily movement can be used to obtain a signal representing a sleep state of the individual; for example, via a respiratory movement of the individual.

The microphone 140 outputs audio data that can be stored in the memory device 114 and/or analyzed by the processor 112 of the control system 110. The audio data generated by the microphone 140 is reproducible as one or more sound(s), such as sounds collected while the individual is engaging in an activity (e.g., grunting sounds while the individual rises from a seated position and/or sounds of apneas during the individual's sleep). The audio data form the microphone 140 can also be used to identify (e.g., using the control system 110) an event experienced by the individual, such as an event experienced during a sleep session. In some cases, microphone 140 can be positioned within the environment, such as in a housing positioned within the environment or mounted to a wall or ceiling of the environment. In some cases, the microphone 140 can be coupled to or integrated in the respiratory device 122, the user interface 124, the conduit 126, the user device 170, or the like. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the user device 170 (e.g., the microphone 140 is a microphone of a smart phone). The microphone 140 can be integrated into the user interface 124, the conduit 126, the respiratory therapy device 122, or any combination thereof.

The speaker 142 outputs sound waves, which may be audible to an individual and/or user of the system 100. In some cases, the sound waves can be inaudible to human ears (e.g., ultrasonic sound waves). The speaker 142 can be used, for example, as an alarm clock or to play an alert or message to an individual or user (e.g., in response to an event or to convey information, such as context-specific insights). In some implementations, the speaker 142 can be used to communicate the audio data generated by the microphone 140 to an individual or user. The speaker 142 can be coupled to or integrated in the respiratory device 122, the user interface 124, the conduit 126, the user device 170, or the like.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the individual. Based at least in part on the data from the microphone 140 and/or the speaker 142, the control system 110 can determine a location of the individual and/or one or more parameters (e.g., sleep-related parameters) associated with the individual, such as, for example, a respiration signal, a respiration rate, an inspiration amplitude, an expiration amplitude, an inspiration-expiration ratio, a number of events (e.g., apneas) per hour, a pattern of events, a sleep stage, pressure settings of the respiratory therapy device 122, a mouth leak status, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, ultra wideband (UWB) signals, millimeterwave (mmWave) signals, etc.). The RF receiver 146 detects the reflections of the radio waves emitted from the RF transmitter 148, and this data can be analyzed by the control system 110 to determine a location of an individual and/or one or more parameters associated with the individual. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are depicted as being separate and distinct elements, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine movement data, position data, one or more parameters associated with the individual, or the like. For example, the image data from the camera 150 can be used to identify a location of an individual, such as to determine a time when the individual sits in a chair and a time when the individual exits the chair, or to identify a path the individual takes when walking from point A to point B in an environment.

The infrared (IR) sensor 152 outputs infrared image data reproducible as one or more infrared images (e.g., still images, video images, or both) that can be stored in the memory device 114. The infrared data from the IR sensor 152 can be used to determine movement data, position data, one or more parameters associated with the individual (e.g., a temperature of the individual), or the like. The IR sensor 152 can also be used in conjunction with the camera 150 when measuring the presence, location, and/or movement of the individual. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the individual that can be used to determine one or more parameters associated with the individual, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the individual, embedded in clothing and/or fabric that is worn by the individual, embedded in and/or coupled to the user interface 124 and/or its associated headgear (e.g., straps, etc.), etc.

The ECG sensor 156 outputs physiological data associated with electrical activity of the heart of the individual. In some implementations, the ECG sensor 156 includes one or more electrodes that are positioned on or around a portion of the individual. The physiological data from the ECG sensor 156 can be used, for example, to determine one or more parameters associated with the individual.

The EEG sensor 158 outputs physiological data associated with electrical activity of the brain of the individual. In some implementations, the EEG sensor 158 includes one or more electrodes that are positioned on or around the scalp of the individual. The physiological data from the EEG sensor 158 can be used, for example, to determine a sleep state of the individual at any given time during a sleep session. In some implementations, the EEG sensor 158 can be integrated in a user interface 124 and/or its associated headgear (e.g., straps, etc.).

The capacitive sensor 160, the force sensor 162, and the strain gauge sensor 164 output data that can be stored in the memory device 114 and used by the control system 110 to determine movement data, position data, one or more parameters described herein, or the like. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the individual (e.g., a therapy user making use of the respiratory system 120). The data output by the analyte sensor 174 can be stored in the memory device 114 and used by the control system 110 to determine the identity and concentration of any analytes in the breath of the therapy user. In some implementations, the analyte sensor 174 is positioned near a mouth of the therapy user to detect analytes in breath exhaled from the therapy user's mouth. For example, when the user interface 124 is a face mask that covers the nose and mouth of the therapy user, the analyte sensor 174 can be positioned within the face mask to monitor the therapy user's mouth breathing. In other implementations, such as when the user interface 124 is a nasal mask or a nasal pillow mask, the analyte sensor 174 can be positioned near the nose of the therapy user 210 to detect analytes in breath exhaled through the therapy user's nose. In still other implementations, the analyte sensor 174 can be positioned near the therapy user's mouth when the user interface 124 is a nasal mask or a nasal pillow mask. In this implementation, the analyte sensor 174 can be used to detect whether any air is inadvertently leaking from the therapy user's mouth. In some implementations, the analyte sensor 174 is a volatile organic compound (VOC) sensor that can be used to detect carbon-based chemicals or compounds. In some implementations, the analyte sensor 174 can also be used to detect whether the therapy user is breathing through their nose or mouth. For example, if the data output by an analyte sensor 174 positioned near the mouth of the therapy user or within the face mask (in implementations where the user interface 124 is a face mask) detects the presence of an analyte, the control system 110 can use this data as an indication that the therapy user is breathing through their mouth.

The moisture sensor 176 outputs data that can be stored in the memory device 114 and used by the control system 110. The moisture sensor 176 can be used to detect moisture in various areas surrounding the individual (e.g., within the environment, on or around a piece of furniture in the environment, on the individual, inside the conduit 126 or the user interface 124, near the individual's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the ambient humidity of the environment surrounding the individual, for example, the air inside the bedroom.

The Light Detection and Ranging (LiDAR) sensor 178 can be used for depth sensing, such as to determine movement data and/or position data. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While depicted as separate components, any combination of the one or more sensors 130 can be integrated in and/or coupled to any one or more of the components of the system 100, including the respiratory device 122, the user interface 124, the conduit 126, the humidification tank 129, the control system 110, the user device 170, the wearable device 190, or any combination thereof. For example, the microphone 140 and speaker 142 can be integrated in and/or coupled to the user device 170 and the pressure sensor 130 and/or flow rate sensor 132 can be integrated in and/or coupled to the respiratory device 122. In some implementations, at least one of the one or more sensors 130 is not coupled to the respiratory device 122, the control system 110, the user device 170, or the wearable device 190, and is positioned elsewhere within the environment, such as on a wall or ceiling of the environment or on a piece of furniture within the environment.

In an example, one or more of the sensors 130 can be located in a first position on a nightstand adjacent to a bed and the individual. Alternatively or in addition, one or more of the sensors 130 can be located in a second position on and/or in a mattress (e.g., the sensor is coupled to and/or integrated in a mattress). Further, one or more of the sensors 130 can be located in a third position on a piece of furniture (e.g., coupled to and/or integrated in a headboard, a footboard, or other location on the frame of the bed, or resting on a nightstand or table). One or more of the sensors 130 can also be located in a fourth position on a wall or ceiling within the environment. The one or more of the sensors 130 can also be located in a fifth position such that the one or more of the sensors 130 is coupled to and/or positioned on and/or inside a housing of the respiratory device 122 of the respiratory system 120. Further, one or more of the sensors 130 can be located in a sixth position such that the sensor is coupled to and/or positioned on the individual (e.g., the sensor(s) is embedded in or coupled to fabric or clothing worn by the individual or present on a wearable device 190 worn by the individual).

The user device 170 includes a display device 172. The user device 170 can be, for example, a mobile device such as a smart phone, a tablet, a laptop, a desktop computer, or the like. In some cases, user device 170 is a device intended to be used primarily by the individual being monitored (e.g., a smartphone of the individual). In some cases, however, user device 170 can be a device intended to be used primarily by a user monitoring the individual (e.g., a desktop computer or tablet used by a caregiver to monitor multiple residences in an assisted living facility). In some cases, the user device 170 can be an external sensing system, a television (e.g., a smart television) or another smart home device (e.g., a smart speaker(s) such as Google Home™, Google Nest™, Amazon Echo™, Amazon Echo Show™, Alexa™-enabled devices, etc.). In some implementations, wearable device 190 is a type of user device 170. Wearable device 190 can be any suitable body-worn device, such as a smart watch, a fitness tracker, or the like. The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human individual interacting with the user device 170. In some implementations, one or more user devices can be used by and/or included in the system 100, such as a separate user device for each user and each individual associated with system 100.

In some cases, wearable device 190 is an activity tracker that can be generally used to aid in generating physiological data for determining an activity measurement associated with the individual. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum the respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. An activity tracker wearable device (e.g., smart watch, wristband, ring, patch, and the like) can include one or more of the sensors 130 described herein, such as, for example, the motion sensor 138 (e.g., one or more accelerometers and/or gyroscopes), the PPG sensor 154, and/or the ECG sensor 156.

While the control system 110 and the memory device 114 are described and depicted as being a separate and distinct component of the system 100, in some implementations, the control system 110 and/or the memory device 114 are integrated in the user device 170, the respiratory device 122, and/or one of the one or more sensors 130. Alternatively, in some implementations, the control system 110 or a portion thereof (e.g., the processor 112) can be located in a cloud (e.g., integrated in a server, integrated in an Internet of Things (IoT) device (e.g., a smart TV, a smart thermostat, a smart appliance, smart lighting, etc.), connected to the cloud, be subject to edge cloud processing, etc.), located in one or more servers (e.g., remote servers, local servers, etc.), or any combination thereof.

While system 100 is shown as including all of the components described above, more or fewer components can be included in a system for generating quantitative scores and providing context-specific insights according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory system 120, at least one of the one or more sensors 130, and a user device 170. Thus, various systems can be formed using any portion or portions of the components shown and described herein and/or in combination with one or more other components.

As used herein, a sleep session can be defined in a number of ways based on, for example, an initial start time and an end time. A sleep timeline can include an enter bed time (t_{bed}), a go-to-sleep time (t_{GTS}), an initial sleep time (tₛₗₑₑₚ), any number of micro-awakenings, a wake-up time (t_{wake}), and a rising time (tᵣᵢₛₑ).

In some implementations, a sleep session is a duration where the individual is asleep. In such implementations, the sleep session has a start time and an end time, and during the sleep session, the individual does not wake until the end time. That is, any period of the individual being awake is not included in a sleep session. From this first definition of sleep session, if the individual wakes ups and falls asleep multiple times in the same night, each of the sleep intervals separated by an awake interval is a sleep session.

Alternatively, in some implementations, a sleep session has a start time and an end time, and during the sleep session, the individual can wake up, without the sleep session ending, so long as a continuous duration that the individual is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the individual first entered the bed, and the time the next morning when individual last left the bed. Put another way, a sleep session can be defined as a period of time that begins on a first date (e.g., Thursday, Jan. 6, 2022) at a first time (e.g., 10:00 PM), that can be referred to as the current evening, when the individual first enters a bed with the intention of going to sleep (e.g., not if the individual intends to first watch television or play with a smart phone before going to sleep, etc.), and ends on a second date (e.g., Friday, Jan. 7, 2022) at a second time (e.g., 7:00 AM), that can be referred to as the next morning, when the individual first exits the bed with the intention of not going back to sleep that next morning.

In some implementations, the individual can manually define the beginning of a sleep session and/or manually terminate a sleep session. For example, the individual can select (e.g., by clicking or tapping) a user-selectable element that is displayed on the display device 172 of the user device 170 to manually initiate or terminate the sleep session.

The enter bed time t_{bed} is associated with the time that the individual initially enters the bed prior to falling asleep (e.g., when the individual lies down or sits in the bed). The enter bed time t_{bed} can be identified based on a bed threshold duration to distinguish between times when the individual enters the bed for sleep and when the individual enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the individual initially enters any location for sleeping (e.g., a couch, a chair, a sleeping bag, etc.).

The go-to-sleep time (GTS) is associated with the time that the individual initially attempts to fall asleep after entering the bed (t_{bed}). For example, after entering the bed, the individual may engage in one or more activities to wind down prior to trying to sleep (e.g., reading, watching TV, listening to music, using the user device 170, etc.). The initial sleep time (tₛₗₑₑₚ) is the time that the individual initially falls asleep. For example, the initial sleep time (tₛₗₑₑₚ) can be the time that the individual initially enters the first non-REM sleep stage.

The wake-up time t_{wake} is the time associated with the time when the individual wakes up without going back to sleep (e.g., as opposed to the individual waking up in the middle of the night and going back to sleep). The individual may experience one of more unconscious microawakenings having a short duration (e.g., 5 seconds, 10 seconds, 30 seconds, 1 minute, etc.) after initially falling asleep. In contrast to the wake-up time t_{wake}, the individual goes back to sleep after each of the microawakenings. Similarly, the individual may have one or more conscious awakenings after initially falling asleep (e.g., getting up to go to the bathroom, attending to children or pets, sleep walking, etc.). However, the individual goes back to sleep after the awakening. Thus, the wake-up time t_{wake} can be defined, for example, based on a wake threshold duration (e.g., the individual is awake for at least 15 minutes, at least 20 minutes, at least 30 minutes, at least 1 hour, etc.).

Similarly, the rising time tᵣᵢₛₑ is associated with the time when the individual exits the bed and stays out of the bed with the intent to end the sleep session (e.g., as opposed to the individual getting up during the night to go to the bathroom, to attend to children or pets, sleep walking, etc.). In other words, the rising time tᵣᵢₛₑ is the time when the individual last leaves the bed without returning to the bed until a next sleep session (e.g., the following evening). Thus, the rising time tᵣᵢₛₑ can be defined, for example, based on a rise threshold duration (e.g., the individual has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the individual has left the bed for at least 4 hours, at least 6 hours, at least 8 hours, at least 12 hours, etc.).

As described above, the individual may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the individual. For a standard individual who goes to bed in the evening, then wakes up and goes out of bed in the morning any period (between the individual waking up (t_{wake}) or raising up (tᵣᵢₛₑ), and the individual either going to bed (t_{bed}), going to sleep (t_{GTS}) or falling asleep (tₛₗₑₑₚ) of between about 12 and about 18 hours can be used. For individual that spend longer periods of time in bed, shorter threshold periods may be used (e.g., between about 8 hours and about 14 hours). The threshold period may be initially selected and/or later adjusted based on the system monitoring the individual's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, the total sleep time (TST) can span between the initial sleep time tₛₗₑₑₚ and the wake-up time t_{wake}, but excludes the duration of any micro-awakenings or awakenings. Thus, the total sleep time (TST) may be shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the individual is initially falling asleep, the individual may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

In some cases, time spent engaging in other activities can be similarly defined, such as based on times when the individual intends to begin engaging in the activity, when the individual actually begins engaging in the activity, excluding times when the individual temporarily disengages with the activity, and further based on times when the individual ceases to engage with the activity. For example, for the activity of sitting in a chair, the system 100 can determine the time the individual first intended to sit in the chair (e.g., the individual initiates a first attempt to sit in the chair), determine the time the individual first actually begins engaging in the activity (e.g., the individual initiates a successful attempt to sit in the chair), determine the time the individual ceases to engage in the activity (e.g., the individual stands up), the time spent attempting to sit in the chair (e.g., the time between initially attempting to sit in the chair and actually completing sitting in the chair), the time spent attempting to exit the chair (e.g., the time between initially attempting to exit the chair and the time when the individual is standing), and other such parameters.

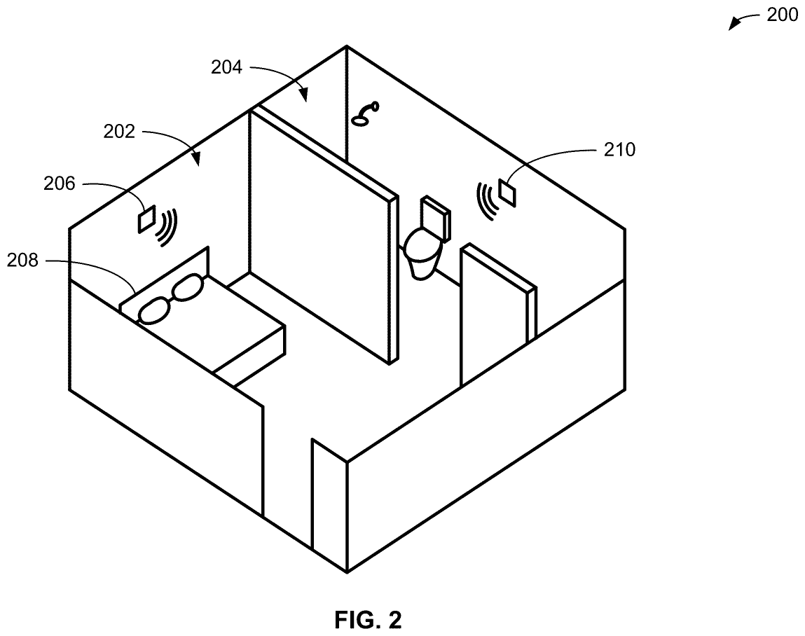

FIG. 2 is an isometric view of an environment 200 which may be occupied by an individual for which a quantitative health score is generated, according to certain aspects of the present disclosure. In some cases, the environment 200 can be a set of rooms in a larger environment (e.g., a set of rooms within an assisted living facility or set of rooms within a larger house). While many different types of environments can be used, FIG. 2 depicts an example of an environment 800 that includes a bedroom 202 and a bathroom 204.

In some cases, the system can use sensors 206, 210 that are radar-based sensors. Radar sensors can be used to identify various parameters that are usable to determine routines and determine deviations from routines. Such parameters can include (i) detecting when an individual falls; (ii) detecting when an individual is in a room; (iii) detecting when an individual enters a sub-region or zone within a room (e.g., an area next to a bathroom or next to a bed); (iv) detecting when the individual is moving in a room; (v) detecting when and/or how the individual is breathing; (vi) detecting a time spent in bed; (vii) detecting a time spent sleeping and/or a time spent in one or more sleep stages; (viii) detecting when an individual visits the bathroom; (ix) detecting when an individual wanders out of bed and/or out of their bedroom at night; (x) detecting an amount of time the individual spends outside of bed; (xi) detecting a pose of the individual; (xii) detecting an individual's gait; (xiii) detecting a walking speed of the individual; (xiv) detecting presence of a visitor; (xv) detecting location of an individual at a given point in time to build a map of the individual's location in a room over time; or (xvi) any combination of (i)-(xv).

Bedroom 202 can include a bed 208 and optionally other furniture. A bedroom sensor 206 can be positioned within the bedroom 202, such as on a wall of the bedroom 202. In some cases, however, sensor 206 can be positioned outside of the bedroom 202 but positioned and/or configured to collect data about the bedroom 208.

Environment 200 can further include a bathroom 204 connected to the bedroom 202 by an entrance (e.g., a doorway). A second sensor 210 can be positioned within bathroom 202, although that need not always be the case.

In this example, an individual living in the environment 200 can move around within the bedroom 202 and bathroom 204 while being monitored by sensors 206, 210. Sensors 206, 210 can be any suitable sensor (e.g., any of one or more sensors 130 of FIG. 1). In some cases, sensors 206, 210 can be radar sensors that are able to monitor the position and/or movements of the individual throughout the environment 200 in a privacy-maintaining fashion (e.g., without capturing the individual using visible light).

For example, as the individual moves towards the bathroom 204, sensor 206 can identify that the individual is moving toward the bathroom 204 and infer that the individual is going to use the bathroom, such as to use the toilet, take a bath or shower, or otherwise maintain the individual's personal hygiene. When the individual is finished, the sensor 206 may identify that the individual is returning from the bathroom 204. Thus, the system can monitor how long the individual spends in the bathroom 204 and/or how often the individual visits the bathroom 204. In some cases, a sensor 210 in the bathroom 204 can be used to further differentiate what actions the individual is taking within the bathroom 204. While the individual moves around the bedroom 202, the sensor 206 can monitor the individual performing various actions, such as siting and standing, walking from point A (e.g., beside the bed 208) to point B (e.g., at the doorway leading out of the bedroom 202), sleeping, and the like.

Environment 200 can include any suitable number of rooms, with any suitable number of sensors.

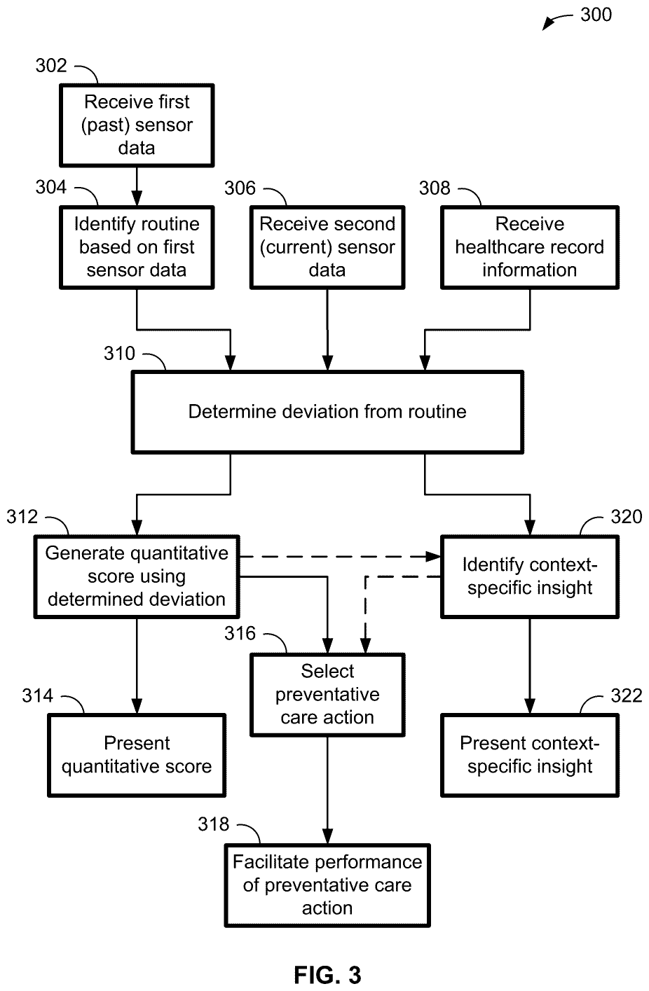

FIG. 3 is a flowchart depicting a process 300 for generating a quantitative health score, according to certain aspects of the present disclosure. Process 300 can be performed by system 100 of FIG. 1, such as in environment 200 of FIG. 2.

At block 302, first sensor data is received. First sensor data can also be known as past sensor data. The first sensor data is received by one or more sensors (e.g., one or more sensors 130 of FIG. 1). The first sensor data can be collected over a period of time, such as a period of minutes, hours, days, weeks, months, years, or the like. As used herein, first sensor data, or past sensor data, can refer to all sensor data used to identify a routine at block 304. For example, while second sensor data from block 306 is initially collected to determine a deviation from a routine, as described in further detail herein, if that second sensor data is subsequently used to identify a routine (e.g., to update an existing routine based on that new sensor data), that new sensor data can be considered “past” sensor data for purposes of the updated routine and any actions taken with respect to the updated routine (e.g., determination of deviations from that updated routine).

At block 304, one or more routines are identified based on the first sensor data from block 304. Identification of a routine can occur in any suitable fashion, such as those described herein. In some cases, identification of a routine includes applying one or more rules and/or a trained machine learning algorithm to the received first sensor data from block 302 to identify the routine. In some cases, a set of available routines exist which the system is able to identify. In some case, the set of available routines can be based on the type(s) of sensor(s) used to collect the sensor data from block 302, and/or the locations of the sensor(s). For example, a first set of available routines can exist if the only sensor is a radar sensor located within a room in the environment, this first set of available routines including routines such as time to walk from point A to Point B, path used to walk from point A to Point B, number of visits to a bathroom, and the like. In that example, a second set of available routines can exist if the individual makes use of a wearable device able to track heart rate, heart rate variability, blood oxygenation, and other such data. In such cases, the set of available routines can include an average heart rate while engaging in a particular activity (e.g., walking from point A to point B) or an average blood oxygenation while sleeping. Other routines can be used. In some cases, available routines can be preset by a clinician or other caregiver. In some cases, a machine learning algorithm can automatically identify routines based on the first sensor data.

At block 306, second sensor data is received. Second sensor data can also be known as current sensor data. The second sensor data is received by the same one or more sensors used to collect the first sensor data from block 302. The second sensor data can be compared to the first sensor data to determine whether a deviation from an identified routine exists. The second sensor data is collected after the first sensor data, and can be collected over a period of time, such as a period of minutes, hours, days, weeks, months, years, or the like. For example, second sensor data collected over a period of minutes or hours may be used to identify deviations that occurred in a particular day (e.g., “the individual is having more difficulty walking today than in the past few weeks”), whereas second sensor data collected over a period of days or weeks may be used to identify deviations that occurred in the past several days or weeks (e.g., “the individual has been spending more time in the bathroom in the past couple weeks than in the past several months”).

At block 310, one or more deviations from the routine(s) identified at block 304 can be determined. One or more routines identified at block 304 can be accessed and used as baselines for comparison with respective values interpreted from the second sensor data. At block 310, the second sensor data can be analyzed to determine whether this current sensor data fits within the identified routine(s) from block 304 or strays from the identified routine(s) by more than a threshold amount. The output of block 310 can be binary in nature (e.g., a sufficient deviation exists or does not exist), can be tertiary in nature (e.g., no sufficient deviation exists, a sufficient positive deviation exists, or a sufficient negative deviation exists), or can be specific in nature (e.g., a specific value of the deviation and/or a specific z-score associated with the deviation).

In an example, first sensor data received at block 302 can include movement data collected over the course of multiple weeks. From this first sensor data, block 304 identifies as a routine an average path taken by the individual when the individual goes from the bed to the bathroom at night. This routine path can be represented as a collection of data points indicating subsequent locations in the environment as the individual moves from the bed to the bathroom. At any time after the routine has been identified at block 304, such as the following night, second sensor data can be collected and used to identify the current path taken by the individual on a current night. Then, at block 310, a determination can be made that the current path deviates from the routine path by a certain amount. In some cases, if the amount of deviation is minimal (e.g., below a threshold value, such as having a z-score at or below a certain value or within a range including zero), the current path will be considered to not be a deviation from the routine. However, if the amount of deviation is sufficiently high (e.g., above a threshold value, such as having a z-score at or above a certain value, or outside of a range including zero), it can be determined that a deviation has occurred. Block 310 can then output either (i) an indication that a deviation has occurred for a particular routine; (ii) an indication a direction of a deviation that has occurred for the particular routine; (iii) an indication of the amount of deviation that has occurred for the particular routine; or (iv) any combination of (i)-(iii).

In some cases, healthcare record information can be received at block 308. Healthcare record information can include any healthcare information that is accessible by the system, such as healthcare information stored in a memory of the system or otherwise accessible to the system, such as via a network connection to an EHR database. In some cases, receiving healthcare record information at block 308 can specifically entail requesting healthcare record information associated with the identified routine(s) from block 304. For example, if a routine identified at block 304 entails a time spent going to the bathroom, receiving healthcare record information at block 308 can include requesting certain healthcare record information associated with going to the bathroom (e.g., listing of prescription medications that may affect time spent going to the bathroom or listing of diagnoses that may affect time spent going to the bathroom). In some cases, identifying a routine at block 304 is optionally based at least in part on received healthcare information from block 308, such as historical (e.g., past) healthcare information.

In some cases, determining a deviation at block 310 can further be based at least in part on the received healthcare record information. In some cases, healthcare record information can be used to inform whether or not a perceived deviation from a routine is expected or unexpected based on the healthcare record information. For example, if a routine associated with time spent going to the bathroom is established and the current sensor data shows that the individual has been taking longer and longer to go to the bathroom in recent days, block 310 may normally determine that a deviation has occurred. However, if received healthcare record information shows that the individual has recently began taking diuretic medication, block 310 may determine that a deviation from routine has not occurred because the amount of the deviation is within an expected threshold range expected for the individual after beginning to take the diuretic medication. In some cases, the healthcare record information can be used to dynamically adjust threshold values or ranges used to determine deviations from routines.

At block 312, a quantitative score can be generated using the determined deviation from block 310. In some cases, determining the quantitative score can further include using received sensor data from block 302, received sensor data from block 306, received healthcare record information from block 308, and/or any combination thereof.

A quantitative score can be based at least in part on the determined deviation from the routine. The value of the determined deviation (e.g., an amount of deviation) and/or a presence of the determined deviation (e.g., a binary 1 or 0 indicating presence or absence of a deviation, respectively) can be used to determine and/or adjust the quantitative score.

In some cases, a quantitative score can be generated by applying a trained machine learning algorithm to input data (e.g., determined deviations as output from block 310) to output a quantitative score.

In some cases, the quantitative score can be generated by combining multiple component scores, optionally with respective weighting values. For example, a “total health score” can be generated from a combination of component scores including at least two of a mobility score, a sleep score, a social score, a physical score, a respiratory/cardiovascular score, and a mental health score. Each component score can be generated based on one or more determined deviations from block 310, received sensor data from block 302, received sensor data from block 306, received healthcare record information from block 308, and/or any combination thereof. In some cases, a component score can itself be comprised of multiple sub-component scores. For example, a mobility component score may be based at least in part on a fall frequency score and an activity score, the former of which is indicative of the frequency that the individual has fallen in a past period of time, the latter of which is indicative of the individual's level of activity.

In some cases, a quantitative score generated from multiple component scores can include the use of at least a mobility score and a sleep score. The use of at least a mobility score and at least a sleep score can be especially useful to monitor the overall health of an individual, especially individuals in assisted living environments or certain healthcare environments. The mobility score is indicative of the individual's ability to move around within the environment, which can be important to or otherwise informative of many aspects of the individual's health (e.g., movement can be important to maintaining social activity and can be an early indicator of declining mental health), and the sleep score is indicative of the individual's ability to rest and recover through sleep, which can also be important or otherwise informative to many aspects of the individual's health (e.g., ability to sleep can be indicative of pain experienced by the individual and/or a mental state of the individual). Nevertheless, in some cases, other components scores can be used in addition to or instead of a mobility score and a sleep score.

In some cases, component scores are weighted according to respective weighting values. The weighting values can be initially preset to a default value. In some cases, one or more weighting values can be adjusted by a clinician or other healthcare professional. In some cases, the one or more weighting values can be adjusted by a user and/or the individual. In some cases, the weighting values can be adjusted dynamically, such as based on one or more historical quantitative score(s) and/or historical component score(s), based on one or more current and/or historical determined deviations, based on the first sensor data, based on the second sensor data, based on the one or more identified routines, based on the received healthcare record information, or any combination thereof.

In some cases, certain component scores and/or sets of component scores can be weighted more strongly than other component scores and/or sets of component scores. For example, in some cases, each component score can be assigned an importance level, which can affect how strongly that component score is weighted.

In an example, a quantitative score can be generated from component scores including a fall score (e.g., score indicative of a number of falls in a recent period of time), an activity score (e.g., a score indicative of the individual's level of activity), a sleep score (e.g., a score indicative of the individual's sleep performance and/or respirator usage), a bathroom usage score (e.g., a score indicative of a frequency of bathroom use or time spent using the bathroom), a personal hygiene score (e.g., a score indicative of a frequency of or time spent maintaining personal hygiene, such as showering), an infection risk score (e.g., a score indicative of a risk of infection, such as through analysis of respiration rate, time spent in bed (duration and/or time of day), sedentary time, bathroom usage (frequency and/or duration), medication usage (e.g., antibiotics), recent hospitalization, visitor history and contact tracing history, and the like), a physical/mobility score (e.g., a score indicative of the individual's physical ability or mobility, such as through analysis of sit/stand duration, walking speed, walking duration, sedentary time, fall history, personalized gait pattern, previous orthopedic procedures, medication history (e.g., blood pressure medications), and the like), and a cognitive score (e.g., a mental health score, such as through analysis of sundowning (e.g., excessive activity and/or wandering at sunset), hygiene patterns, repeated activities (e.g., bathroom recurrence in short time periods), sleep patterns, night wandering, social connectedness, and the like). In this example, the fall score and the infection score can receive an importance level of “1” and the other scores can receive an importance level of “2.” The scores with an importance level of 1 will be considered of higher importance and will receive a stronger weighting than those with an importance level of 2. Other importance levels can be used.

At block 314, the quantitative score can be presented. Presenting a quantitative score can occur as disclosed herein, such as by presenting the quantitative score as a number on a display device (e.g., display device 172 of FIG. 1). In some cases, presenting the quantitative score can include presenting the quantitative score as a percentage or as a value out of 100. In some cases, presenting the quantitative score can include presenting a partially filled-in shape (e.g., a ring or other shape) that is filled in with the same percentage as the quantitative score (e.g., a score of 75 out of 100 or 3 out of 4 can be represented by a shape that is 75% filled in).

In some cases, presenting a quantitative score at block 314 can include presenting one or more historical quantitative scores, such as in a fashion that allows for quick and easy comparison between the historical quantitative score(s) and the current quantitative score.

In some cases, presenting a quantitative score at block 314 can include presenting the one or more component scores that are used to generate the quantitative score. Presenting a component score can include presenting an indication of how much the component score influenced the quantitative score. For example, a component score with a high weighting value may nevertheless effect the quantitative score more than a component score with a low weighting value, even if the component score with the low weighting value is higher than the component score with the high weighting value.

In some cases, at block 320, one or more context-specific insights can be identified. The identified context-specific insight(s) can be presented at block 322. In some cases, presenting a context-specific insight can include displaying the context-specific insight, optionally in association with a quantitative score or one or more component scores, on a display device.

Context-specific insights can be identified based on one or more current and/or historical deviations from block 310, one or more current and/or historical quantitative score(s) and/or component score(s) from block 312, the first sensor data, the second sensor data, the one or more identified routines, the received healthcare record information, or any combination thereof. In some cases, identification of a context-specific insight can be triggered based on one or more current and/or historical deviations from block 310, one or more current and/or historical quantitative score(s) and/or component score(s) from block 312, the first sensor data, the second sensor data, the one or more identified routines, the received healthcare record information, or any combination thereof.

For example, when a particular deviation from a routine is detected at block 310, identification of a context-specific insight can be triggered. The identification of the context-specific insight can be based on the deviation from the routine and one or more component scores generated based on that deviation.

In another example, when a particular component score from block 312 drops below a threshold level, identification of a context-specific insight can be triggered. The identification of the context-specific insight can be based on any determined deviation(s) from block 310 that led to the drop in the particular component score. In an example, if the system detects a sufficient drop in an infection component score, the system may identify that the individual's average time in bed for the past couple days has been especially high and the individual has been experiencing an especially high respiration rate. The system may identify these factors as context-specific insights that may be related to the drop in the infection component score. In some cases, this context-specific insight can be paired with preventative care action(s) as described below (e.g., an indication that potential therapy may be warranted, a wellness visit may be warranted, a urinary tract infection or pneumonia work up may be warranted, and/or a visit with a caregiver (e.g., primary care physician) and/or hospitalization may be warranted.

In another example, when deviations from the routine determine that the individual has been experiencing an increase in sedentary time not long after contact with a visitor (e.g., family member visiting or contact with another resident in a facility), a context-specific insight may indicate that the individual may be experiencing symptoms associated with a health condition that may have been contracted through the visit (e.g., potential exposure to COVID-19 during a recent visit may have caused the individual to become more sedentary than routine over the past few days).

In some cases, context-specific insights identified at block 320 include i) a duration of time asleep; ii) a duration of time spent in one or more sleep stages; iii) a number of sleep disruptions; iv) a duration of time spent awake after a sleep disruption; v) a room in the environment in which the target individual remains after the sleep disruption; vi) a number of bathroom visits for a given timeframe; vii) a time of bathroom visits; viii) a duration of bathroom visits; ix) a duration of time in bed; x) a duration of time in a sitting position; xi) a start time associated with the duration of time in bed or the duration of time in the sitting position; or xii) any combination of i-xi.

Similarly to how context-specific insights are identified and presented at block 320 and 322, preventative care action(s) can be selected and performance thereof can be facilitated at blocks 316, 318, respectively. Preventative care actions can be selected based on one or more current and/or historical deviations from block 310, one or more current and/or historical quantitative score(s) and/or component score(s) from block 312, the first sensor data, the second sensor data, the one or more identified routines, the received healthcare record information, identified context-specific insights from block 320, or any combination thereof. In some cases, selection of a preventative care action can be triggered based on one or more current and/or historical deviations from block 310, one or more current and/or historical quantitative score(s) and/or component score(s) from block 312, the first sensor data, the second sensor data, the one or more identified routines, the received healthcare record information, identified context-specific insights from block 320, or any combination thereof.

Preventative care actions can be actions that are designed to (i) prevent deterioration of a health condition; (ii) prevent deterioration of a score (e.g., a quantitative score or component score); (iii) improve a health condition; (iv) improve a score (e.g., a quantitative score or component score); (v) or any combination of (i)-(iv). In some cases, preventative care actions can be actions designed to otherwise improve the health of the individual.

Selecting a preventative care action at block 316 can include selecting the preventative care action from a list of possible preventative care actions. Selection can be based on one or more rules and/or a trained machine learning algorithm that has been trained to select appropriate preventative care actions based on the provided inputs.

In some cases, selecting a preventative care action can include determining (e.g., based on a quantitative score, a component score, sensor data, or the like) that a future quantitative score will drop below a threshold value. For example, a steadily declining trend in quantitative score may be indicative that a future quantitative score (e.g., a quantitative score on a future day) is expected to drop below a threshold value. Selecting the preventative care action can be based at least in part on the future quantitative score (e.g., the score itself and/or the date on which the score will occur), and can include selecting a preventative care action designed to improve the future quantitative score.

Facilitating performance of a preventative care action at block 318 can include presenting an alert indicating the preventative care action to be taken (e.g., “Individual should receive a pneumonia screening test”); presenting an alert indicating actions to take to perform the preventative care action (e.g., “Individual should practice sitting and standing using a walker three times this afternoon”); automatically instigating performance of the preventative care action, optionally after receiving user or professional confirmation, (e.g., “A checkup with a nurse practitioner has been scheduled for tomorrow afternoon to assess your respiratory health”), or any combination thereof.

In some cases, context-specific insights and preventative care actions can be presented in concert with another. For example, if a routine time to rise from a sitting position and a routine duration to walk across a room has been established for an individual, if the trend over the past 90 days shows a gradual increase in both times, context-specific insight can be presented indicating that this detected trend and explaining that the trend indicates potential increase in frailty, and preventative care action(s) can be presented indicating that the individual should consider a wellness visit, additional exercise, and/or additional balance training.

In another example, determined deviations in the individual's routine gait patterns may indicate potential early onset dementia and/or Alzheimers. Context-specific insight can be presented indicating the detected deviations and explaining that they may indicate potential early onset dementia and/or Alzheimers, and preventive care action(s) can be presented indicating that the individual should consider a cognitive decline assessment, mental health coaching, and/or planning with family for additional care and/or transition to a memory care unit.

In another example, a system may determine that in the past 60 days, the individual has shown an increase in routine movement between rooms in the environment in the early evening and decrease in the use of the shower. A context-specific insight can be identified and presented indicating these factors and explaining that they may indicate a change in the cognitive state of the individual, and preventative care action(s) can be presented indicating that the individual should consider a wellness visit to assess cognitive state, mental health coaching, and/or additional care in home or transition to a memory care unit.

While process 300 is depicted with certain blocks in a certain order, in some cases, process 300 can include fewer blocks and/or additional blocks, and/or blocks in different orders. For example, in some cases identifying a context-specific insight can occur immediately after receiving second sensor data at block 306.

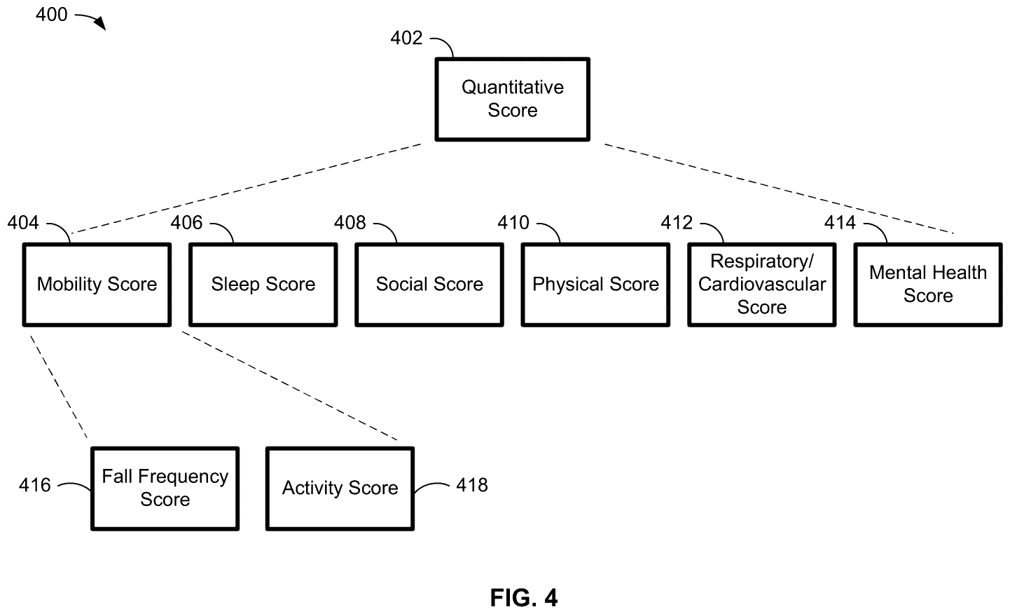

FIG. 4 is a schematic diagram 400 depicting a quantitative score 402 and associated component scores, according to certain aspects of the present disclosure. Quantitative score 402 can be a total health score, such as quantitative score form block 312 of FIG. 3.

The quantitative score 402 can be a single number (e.g., a number from 0 to 100) that is intended to indicate an overall quality of the individual's health across multiple factors.

The quantitative score 402 can be based on a number of individual, component scores. Various combinations of component scores are contemplated as disclosed herein, although one example is depicted in FIG. 4. In this example, the quantitative score 402 is based on a mobility score 404, a sleep score 406, a social score 408, a physical score 410, a respiratory/cardiovascular score 412, and a mental health (e.g., cognitive) score 414. Each of these scores can weighted differently from one another as disclosed herein.

Any of these component scores can be based on multiple sub-component scores. For example, sub-component scores for the mobility score 404 are depicted as being a fall frequency score 416 and an activity score 418. While only the sub-component scores for the mobility score 404 are shown, other component scores may be based on other sub-component scores. In some cases, multiple component scores may be based at least in part on the same sub-component score. For example, in some cases, a fall frequency score 416 may be a sub-component score for both the mobility score 404 and a mental health score 414. Further, in some cases, a component score can be based at least in part on another component score. For example, in some cases, mental health score 414 can be based at least in part on social score 408.

In an example, the mobility score 404 can be based on the individual's activity level (e.g., as identified by the activity score 418), fall frequency (e.g., as identified by fall frequency score 416), and transition ability (e.g., ability to transition from a sitting position to a standing position, such as identified by a deviation from routine).

In an example, the sleep score 406 can be based on the duration of sleep achieved by the individual, the degree of restlessness of the individual during sleep, and the quality of sleep (e.g., time spent in various stages of sleep and/or a subjective assessment of sleep quality).

In an example, a social score 408 can be based on the individual's engagement with others (e.g., number of and duration of visits to/from visitors and/or other residents) and degree of loneliness (e.g., as assessed by number of and duration of visits to/from visitors and/or other residents and/or by an assessment of actions taken by the individual following such visits or leading up to such visits).

In some cases, component scores such as mobility score 404, sleep score 406, and social score 408 can be especially useful (especially in combination) to objectively assess the individual's health and track for potential declines, which can proactively indicate potential health conditions and thus enable a user to provide proactive care to the individual as soon as possible.

In some cases, other component scores, such as a physical score 410, a respiratory/cardiovascular score 412, and a mental health score 414, can be especially useful (especially in combination) to objectively assess various factors of the individual's health that may provide useful insight into the individual's future health. This insight into the individual's future health can allow a caregiver to plan for the future and to take measures to prevent undesired health conditions, avoid further deterioration of health conditions, and/or try and improve health conditions of the individual.

A physical score 410 can be based on the individual's ability to balance, overall frailty, orthopedic healthcare records information, and the like. In some cases, a physical score 410 is based at least in part on a determined deviation, such as i) a change in time to exit a chair; ii) a change in time to sit in a chair; iii) a change in time to cross a room in the environment; iv) a change in time to move from a first point to a second point in the environment; v) a change in gait; or vi) any combination of i-v.

A respiratory/cardiovascular score 412 can be based detection/diagnosis of sleep apnea, detection/diagnosis of pneumonia, detection/diagnosis of congestive heart failure, and the like.

A mental health score 414 can be based on detected/diagnosis of depression, detected agitation, detected/diagnosis of dementia, and the like. In an example, one or more determined deviations from routine(s) can be indicative that an individual's cognitive ability is declining, which can negatively impact the mental health score 414. In some cases, a deviation usable to affect a mental health score 414 can be i) a physical deviation from a routine path, ii) a deviation in time spent engaging in self-hygiene tasks, iii) a deviation in time spent engaging with other individuals in the environment, iv) a deviation in time spent engaging in a pre-defined activity, or v) any combination of i-iv.

In some cases, a score (e.g., a quantitative score 402, a component score, a sub-component score, etc.) can be initialized at an initialization value that is the same for multiple individuals (e.g., the same for all individuals and/or the same for all individuals sharing certain demographic and/or health-related variables). After being initialized, the score can be incremented or decremented based on detection of deviations. Thus, the scores can be relative in nature (e.g., relative to that individual's progress), and not absolute.

For example, when first beginning to use the system, two individuals' mobility scores 404 may be set to 80, regardless of how they compare with one another (e.g., even if the first individual regularly walks more and more easily than the second individual). After a period of time, the system may detect one or more routines associated with mobility score 404 for each individual. Thereafter, if either individual has a positive deviation in a given routine (e.g., a deviation with a Z-score greater than 1), their respective mobility score 404 may be incremented (e.g., by a set amount or by an amount dependent on the value of the deviation). Likewise, if one of the individuals experiences a negative deviation in a given routine (e.g., a deviation with a Z-score below −1), their respective mobility score 404 may be decremented. If no deviation is detected or the deviation is sufficiently small, no change in mobility score 404 would be made. Thus, even if a first individual is much more mobile and active than a second individual, that first individual may have a lower mobility score than the second individual if the first individual's mobility has been declining while the second individual's mobility has been staying the same or declining at a lower rate.

Use of relative scores can be especially useful when the change in a health metric is more important than the comparison of the underlying health metric itself with an ideal standard. For example, it may be more important to know how an individual's mobility changes over time than it is to know how that individual's mobility compares to an ideal standard. Nevertheless, use of relative scores need not always be used. In some cases, an absolute score may be more useful. For example, a fall frequency score may be indicated as a relative score (e.g., a score that increases or decreases based on the individual's frequency of falling), but may be more informative and easier to understand when provided as an absolute score (e.g., a count of the number of instances the individual has fallen in a given timeframe).

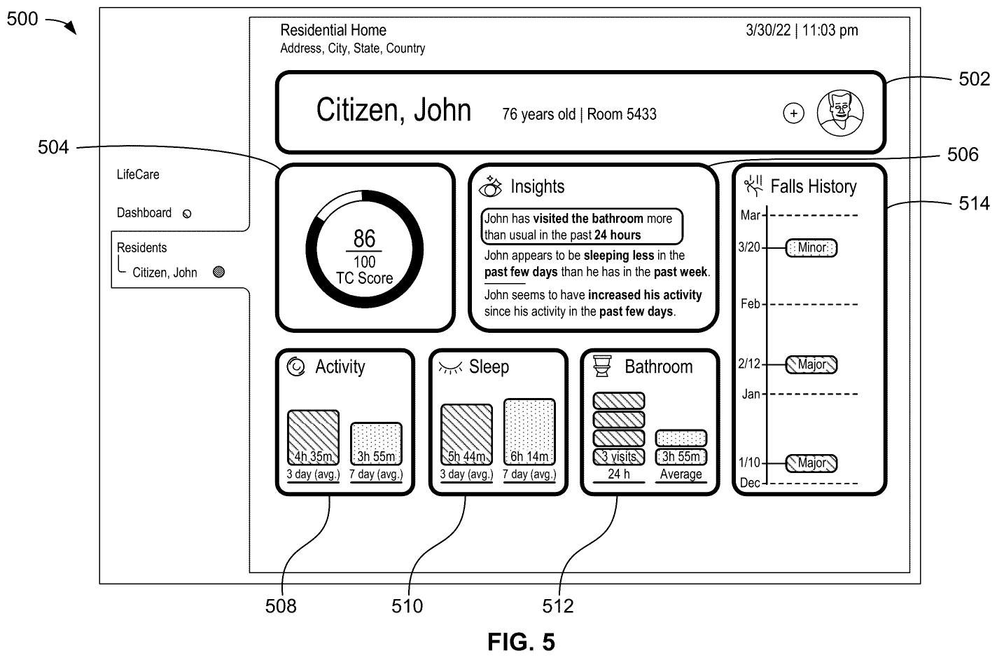

FIG. 5 is a screenshot of an example graphical user interface 500 for viewing a quantitative health score, according to certain aspects of the present disclosure. The GUI 500 can be displayed on any suitable device, such as via display device 172 of FIG. 1.

The GUI 500 can display information about a single individual, although that need not always be the case. The GUI 500 can include a personal information panel 502 that displays personal information about the individual, such as identifying information and/or demographic information about the individual (e.g., the individual's name, age, room number, and an image of the individual).

The GUI 500 can include a quantitative score panel 504 that displays a quantitative score, such as a total health score. The quantitative score can be presented as a partially filled shape (e.g., a ring) that is filled up according to the level of the quantitative score. For example, a score of 100% (e.g., 100 out of 100 or 20 out of 20) would be presented as a fully filled shape, whereas a score of 50% (e.g., 50 out of 100 or 10 out of 20) would be presented as a half-filled shape. In some cases, the score can be additionally presented in text form, optionally with a current value and a maximum value. Other presentation methods can be used, although the described presentation method can be especially useful to allow a user to quickly ascertain the health of the individual.

The GUI 500 can include an insight panel 506. The insight panel 506 can be used to display context-specific insights (e.g., context-specific insights from block 320 of FIG. 3) and/or preventative care actions (e.g., preventative care actions from block 316 of FIG. 3).

The GUI 500 can include one or more component score panels 508, 510, 512, 514. Each component score panel 508, 510, 512, 514 can display a component score and/or other information about a component score (e.g., a sub-component score or other data usable to generate a score). For example, a component score associated with activity, as displayed in component score panel 508, can display a routine (e.g., a 7-day average duration of activity, such as active movement) and a deviation from the routine (e.g., a 3-day average duration of activity that is different than the 7-day average). Similarly, component score panel 510 depicts similar comparisons (e.g., a routine and a deviation from the routine) for sleep (e.g., an average daily duration of sleep). In another example, component score panel 512 depicts a routine (e.g., average number of visits to the bathroom per day) and a deviation from that routine (e.g., the cumulative number of visits to the bathroom this day or within the past 24 hours). In some cases, a component score panel can also depict a routine and an absence of a deviation, such as by depicting that a 7-day average and 3-day average are the same or substantially the same. In the example depicted in component score panel 514, a fall frequency sub-component score is depicted as a listing of falls within the past three months. Further, the listing of falls displays a severity of each fall (e.g., whether the fall was a major fall or a minor fall).

In some cases, the selection of which component score panels 508, 510, 512, 514 to be displayed can be preset, can be user-selectable, and/or can be dynamically selectable. When a component score panel is preset, it will display whatever information was established by the setting. For example, component score panel 514 can be preset to always display information about the individual's fall history. When a component score panel is user-selectable, the user can make a selection to change what is displayed in the component score panel. When a component score panel is dynamically selectable, the system can automatically change what is displayed in the panel based on a quantitative score, a component score (or sub-component score), one or more context-specific insights, one or more preventative care actions, or any combination thereof. As depicted in FIG. 5, component score panels 508, 510, 512 are dynamically selectable, and are automatically adjusted to display information about each of the three insights provided in the insight panel 506.

In some cases, the GUI 500 can provide additional information as well, such as information about the environment (e.g., a name and/or address of the environment, a date, a time, temperature information about the environment, and the like) and links to open other aspects of the GUI 500.

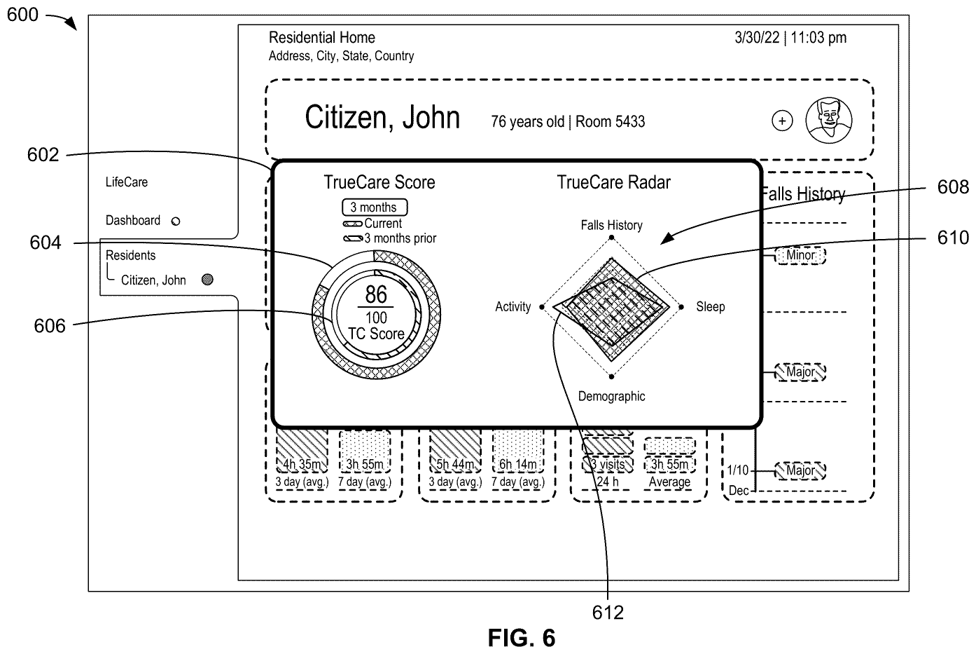

FIG. 6 is a screenshot of an example graphical user interface 600 for comparing current and historical quantitative health scores, according to certain aspects of the present disclosure. The GUI 600 can be displayed on any suitable device, such as via display device 172 of FIG. 1.

The GUI 600 can include a comparison panel 602, which is depicted as a popup or overlay over panels of another GUI (e.g., panels of GUI 500 of FIG. 5), although that need not always be the case.

The GUI 600 can include a current score indicator 604 and a historical score indicator 606. The current score indicator 604 can be a visual representation of a current score, such as a current quantitative score, as described herein. The historical score indicator 606 can be a similar indicator, but for displaying a past, historical score (e.g., a historical quantitative score). The historical score indicator 606 can be de-emphasized with respect to the current score indicator 604. In some cases, such as when the current score indicator 604 and historical score indicator 606 are both partially filled rings, the current score indicator 604 can be a ring with greater diameter, whereas the historical score indicator 606 is a concentric ring with a smaller diameter. In some cases, other visual distinctions can be used additionally or instead, such as the use of different colors to indicate each indicator.

The GUI 600 can include a radar plot 608 for displaying one or more sets of component scores (e.g., a set of current component scores and/or one or more sets of historical component scores). As depicted, radar plot 608 includes a current component scores indicator 610 showing a set of current component scores (e.g., a set of component scores associated with the quantitative score shown by the current score indicator 604) and a historical component scores indicator 612 showing a set of historical component scores (e.g., a set of component scores associated with the historical quantitative score shown by the historical score indicator 606). In some cases, the same or similar visual distinctions (e.g., color distinctions) between the current score indicator 604 and historical score indicator 606 can be used to differentiate the current component scores indicator 610 and the historical component scores indicator 612.

The radar plot 608 can include a separate radial for each type of component score (e.g., a radial for an activity score, a radial for a fall history score, a radial for a sleep score, and a radial for a demographic score (e.g., a score used to evaluate the health of an individual based on demographic information). Each of the component scores indicators 610, 612 can be a shape formed by vertices defined by their respective component scores as plotted on their respective radials. For example, as depicted, the current component scores indicator 610 shows that the current quantitative score, which is indicated by the current score indicator 604, comes from a relatively low activity score, a relatively high falls history score, a relatively high sleep score, and a moderate demographic score; whereas the historical component scores indicator 612 shows that the historical quantitative score, which is indicated by the historical score indicator 606, comes from a relatively high activity score, a relatively low fall history score, a moderate sleep score, and a relatively low demographic score.

Thus, at a quick glance, a user is able to see that the main reasons for why the current quantitative score is improved over the historical quantitative score come primarily from the improved fall history score, improved sleep score, and improved demographic score, although the biggest change appears to be in the fall history score. Further, the radar plot 608 enables a user to quickly determine that while the overall current quantitative score is better than the historical quantitative score, the individual's activity score has dropped and may be in need of improvement.

In some cases, the radar plot 608 can display other information, such as data that is used to generate a component score. Further, while other techniques can be used to display component scores and associated information, the radar plot 608 provides a benefit of showing not only the individual component scores (e.g., as points on the radii), but also showing insight into the overall combined score (e.g., as an area of the shape formed by the points on the radii). Also, this radar plot 608 enables multiple sets of components scores to be easily compared with one another.

In some cases, GUI 600 is GUI 500 after clicking or otherwise interacting with the quantitative score panel 504 of FIG. 5.

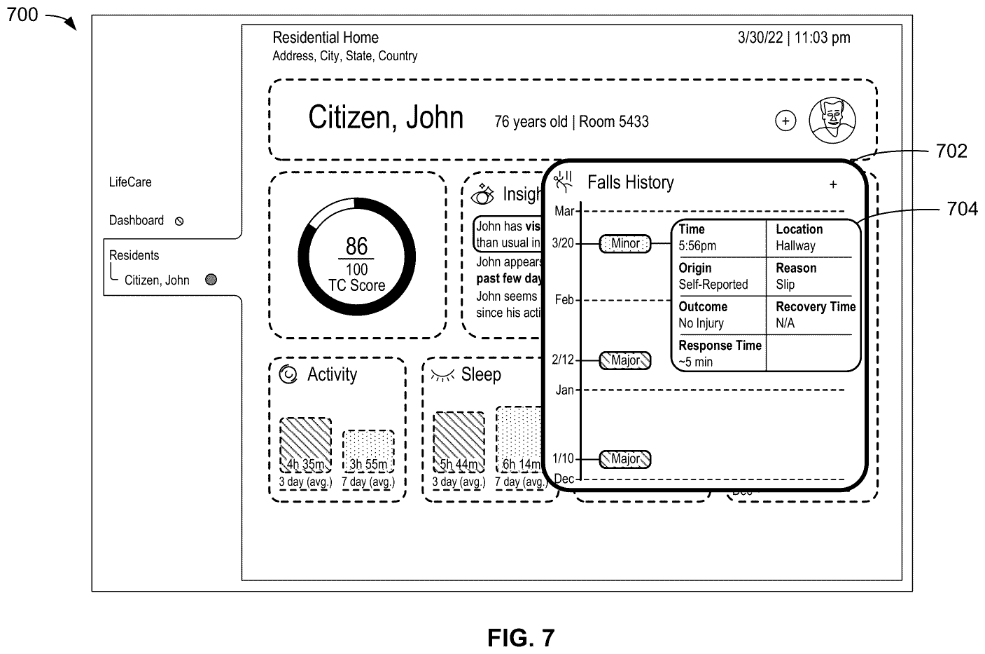

FIG. 7 is a screenshot of an example graphical user interface 700 for viewing an event history associated with a quantitative health score, according to certain aspects of the present disclosure. The GUI 700 can be displayed on any suitable device, such as via display device 172 of FIG. 1.

GUI 700 can include an enlarged component score panel 702, which can display information similar to that from a non-enlarged component score panel (e.g., component score panel 514 of FIG. 5), as well as additional information 704. For example, while component score panel 514 of FIG. 5 depicts fall history information (e.g., information associated with a fall history component score) that includes when falls have occurred and a severity level of the fall, the enlarged component score panel 702 can display additional information 704, such as a time of a fall, a location of a fall, an origin of the fall information (e.g., whether self-reported or automatically detected), a reason for the fall (e.g., slipping, accidentally pushed by another, etc.), an outcome of the fall (e.g., whether any injury occurred), any applicable recovery time (e.g., if an injury occurred, how long it took to recover), and a response time (e.g., an approximate amount of time before a caregiver arrived to assist the individual. Other information could be displayed in addition to or instead of this information.

In some cases, the enlarged component score panel 702 can be displayed in response to clicking or otherwise interacting with a non-enlarged component score panel (e.g., after clicking or otherwise interacting with the component score panel 514 of FIG. 5). Additionally, while depicted as larger in size, an enlarged component score panel 702 need not always appear larger in size.

While the enlarged component score panel 702 is depicted with reference to a fall history component score, any other component score or associated information can be displayed in the enlarged component score panel 702 or a similar enlarged panel. For example, clicking or interacting with the insights panel 506 of FIG. 5 may result in an enlarged version of that panel, with additional information (e.g., more insights or additional information about the displayed insights) being displayed.

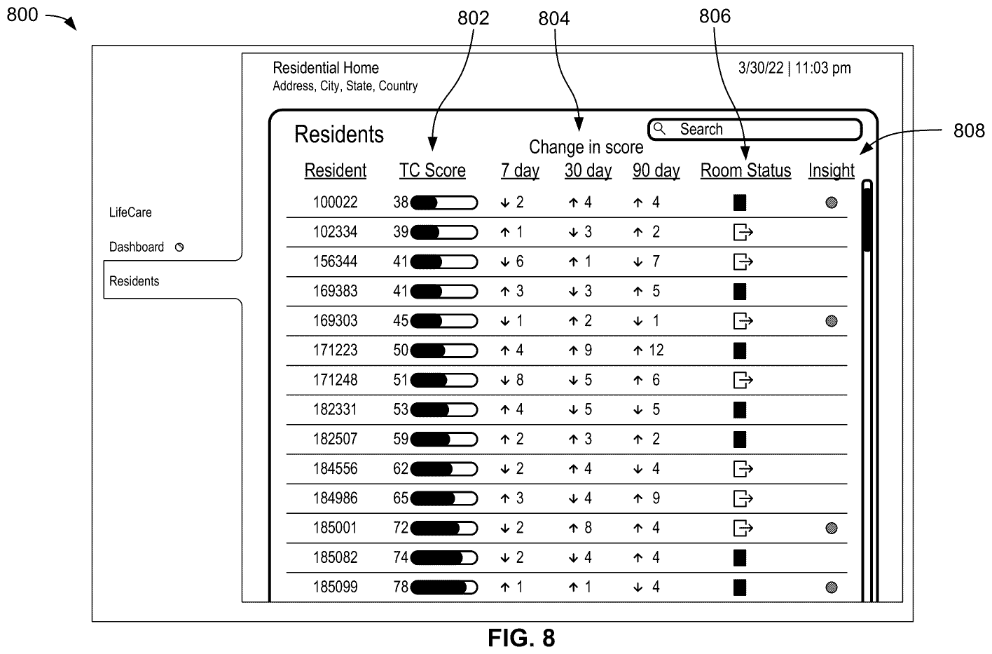

FIG. 8 is a screenshot of an example graphical user interface 800 for viewing quantitative health scores for multiple monitored individuals, according to certain aspects of the present disclosure. The GUI 800 can be displayed on any suitable device, such as via display device 172 of FIG. 1.

GUI 800 can include a listing of multiple individuals (e.g., multiple residents in an assisted living facility), including a listing of their current quantitative scores 802, one or more comparisons 804 between the current quantitative score one or more historical quantitative scores (e.g., a difference between an individual's current quantitative score and their quantitative score from 7 days ago, 30 days ago, and 90 days ago, although other periods can be used), room status information 806 (e.g., an indication of whether the individual is in their bedroom or not), and insight information 808 (e.g., whether or not the system has insight information for review).

The multi-individual GUI 800 allows a user (e.g., caregiver at an assisted living facility) to quickly view and understand the total health scores of multiple individuals under that user's care. Further, the GUI 800 can permit individuals to be sorted by any displayed information, such as sorting the individuals by quantitative score, by comparisons, by room status, or by insights. This ability to sort individuals in this fashion allows users to quickly identify which individuals may need additional care that day, which can help the user triage limited resources (e.g., limited facilities, limited materials, limited instruments, limited personnel, and the like) between the multiple individuals.

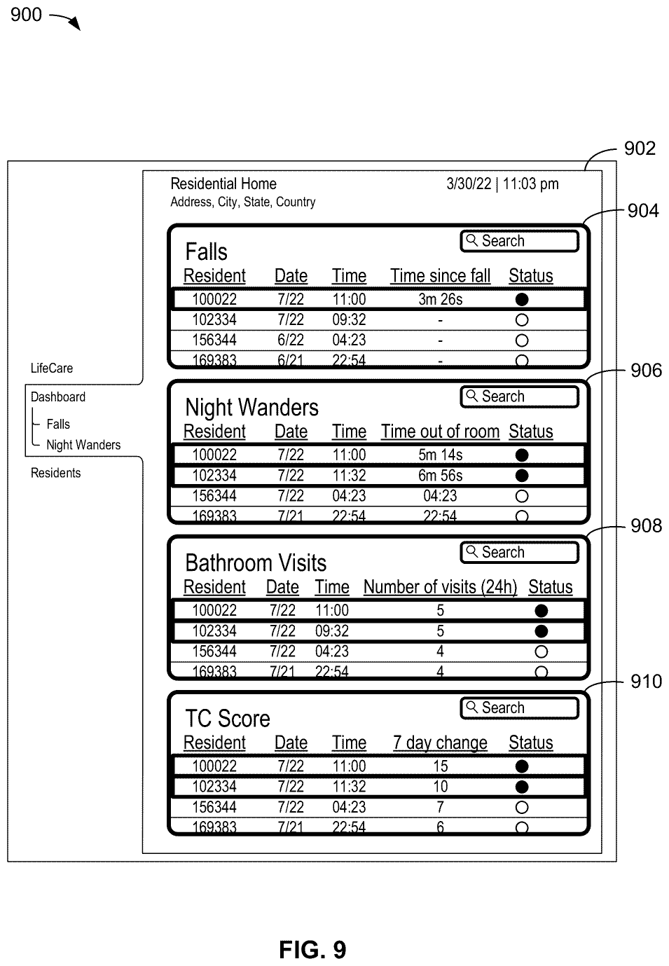

FIG. 9 is a screenshot of an example graphical user interface 900 for viewing select event histories for multiple monitored individuals, according to certain aspects of the present disclosure. The GUI 900 can be displayed on any suitable device, such as via display device 172 of FIG. 1.

The GUI 900 can display a dashboard 902 for monitoring component scores, quantitative scores, and/or other related information for multiple individuals (e.g., multiple residents in an assisted living facility). Dashboard 902 is shown with four panels 904, 906, 908, 910, although any number of panels can be used, each covering any suitable subject (e.g., a particular components score, a quantitative score, or related information).

In an example, panel 904 depicts fall information for a set of individuals. The fall information includes, for each identified individual (e.g., as identified by a resident identifier), a date of the most recent fall, a time of the most recent fall, a time elapsed since the most recent fall (which may be displayed only if below a certain number and/or only while the fall is ongoing, such as until the fall has been addressed), and a status of the fall (e.g., is the fall ongoing or not). With this panel 904, a user can quickly see whether or not an individual has fallen, can quickly identify how long ago the fall occurred, and can quickly identify how long it has been since other individuals have fallen. In some cases, a panel 904 depicting fall information can include a row for each individual, showing that individual's most recent fall information. In some cases, however, panel 904 can include a row for each fall, thus showing multiple rows for a single individual if that individual has fallen multiple times. The panel 904 can be sorted according to any displayed information, such as resident identifier or fall date. In some cases, rows can be highlighted or otherwise emphasized based on status (e.g., ongoing falls are highlighted).

In an example, panel 906 depicts night wandering information for a set of individuals. The night wandering information includes, for each identified individual (e.g., as identified by a resident identifier), a date of the most recent night wandering, a time of the most recent night wandering, a duration of time the individual spent outside of the individual's room, and a status of the night wandering (e.g., is the night wandering ongoing or not). With this panel 906, a user can quickly see whether or not an individual is night wandering, can quickly identify how long the individual has been wandering, and can quickly identify details about past instances of wandering for that individual and/or other individuals. In some cases, a panel 906 depicting night wandering information can include a row for each individual, showing that individual's most recent night wandering information. In some cases, however, panel 906 can include a row for each instance of night wandering, thus showing multiple rows for a single individual if that individual has multiple night wandering incidents. The panel 906 can be sorted according to any displayed information, such as resident identifier or time spent out of the room. In some cases, rows can be highlighted or otherwise emphasized based on status (e.g., ongoing incidents of night wandering are highlighted).

In an example, panel 908 depicts bathroom visit information for a set of individuals. The bathroom visit information includes, for each identified individual (e.g., as identified by a resident identifier), a date of the most recent bathroom visit, a time of the most recent bathroom visit, a number of bathroom visits by that individual within the past 24 hours (or period unit of time), and a bathroom visit status (e.g., is the bathroom visit ongoing or not). With this panel 908, a user can quickly see whether or not an individual is visiting a bathroom, can quickly identify individuals who have an especially large or small number of visits to the bathroom, and other such information. In some cases, a panel 908 depicting bathroom visit information can include a row for each individual, showing that individual's most recent bathroom visit information and 24 hour average. In some cases, however, panel 908 can include a row for each instance of bathroom visit, thus showing multiple rows for a single individual if that individual has multiple bathroom visit incidents, and showing the 24-hour average for the individual in questions as of the time indicated in the row. The panel 908 can be sorted according to any displayed information, such as resident identifier or 24-hour average number of bathroom visits. In some cases, rows can be highlighted or otherwise emphasized based on status (e.g., ongoing visits to the bathroom care highlighted).

In an example, panel 910 depicts total health score information for a set of individuals. The total health score information includes, for each identified individual (e.g., as identified by a resident identifier), a date the total health score was determined, a time the total health score was determined, a comparison metric (e.g., a 7-day change in the total health score), and a total health score status (e.g., is the comparison metric especially large and/or are there available insights and/or preventative care actions that can be reviewed to improve the total health score). With this panel 910, a user can quickly see whether or not an individual's total health score is improving or declining, such as to identify those individuals who have the greatest increase or decline in their total health scores. In some cases, a panel 910 depicting total health score information can include a row for each individual, showing that individual's most recent total health score information and 7-day change. In some cases, however, panel 910 can include a row for each day, thus showing multiple rows for a single individual, and showing the 7-day change as of the given day. The panel 910 can be sorted according to any displayed information, such as resident identifier or 7-day change. In some cases, rows can be highlighted or otherwise emphasized based on status (e.g., individuals with excessive total health score changes can be highlighted).

While certain information is depicted in panels 904, 906, 908, 910, such panels can include other information as disclosed herein, which can facilitate monitoring and caring for one or more individuals.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the disclosure. Thus, the breadth and scope of the present disclosure should not be limited by any of the above described embodiments.

Although certain aspects and features of the present disclosure have been illustrated and described with respect to one or more implementations, equivalent alterations and modifications will occur or be known to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

One or more elements or aspects or steps, or any portion(s) thereof, from one or more of any of claims 1 to 28 below can be combined with one or more elements or aspects or steps, or any portion(s) thereof, from one or more of any of the other claims 1 to 28 or combinations thereof, to form one or more additional implementations and/or claims of the present disclosure.

1. A method comprising:
receiving past sensor data from one or more sensors in an environment, wherein the past sensor data is associated with a target individual in the environment, and wherein the past sensor data is collected over a plurality of past days;
identifying a routine associated with the target individual based at least in part on the past sensor data;
receiving current sensor data from the one or more sensors in the environment, wherein the current sensor data is associated with the target individual in the environment, and wherein the current sensor data is collected after the plurality of past days;
determining a deviation from the routine based at least in part on the identified routine and the received current sensor data;
generating a quantitative score based at least in part on the determined deviation; and
presenting the quantitative score.

2. The method of claim 1, further comprising receiving healthcare record information associated with the target individual, wherein generating the quantitative score is further based at least in part on the received healthcare record information.

3. The method of claim 2, wherein generating the quantitative score includes affecting a weighting of the determined deviation based at least in part on the received healthcare record information.

4. The method of claim 1, further comprising receiving past healthcare record information associated with the target individual, wherein identifying the routine is further based at least in part on the past healthcare record information.

5. The method of claim 1, further comprising:
determining that the deviation is outside of a threshold range;
identifying, based at least in part on at least one of the past sensor data and the current sensor data, one or more context-specific insights associated with the deviation; and
presenting an alert in response to determining that the deviation is outside of the threshold range, wherein presenting the alert includes presenting the one or more context-specific insights.

6. The method of claim 5, wherein determining that the deviation is outside of the threshold range includes determining that the deviation is outside of the threshold range for a threshold duration of time.

7. (canceled)

8. The method of claim 1, wherein presenting the quantitative score further includes presenting a comparison score, wherein the comparison score is a past quantitative score.

9. The method of claim 1, wherein determining the quantitative score includes:
determining a plurality of component scores based at least in part on the determined deviation, the past sensor data, and the current sensor data; and
calculating the quantitative score based on each of the plurality of component scores.

10. The method of claim 9, wherein calculating the quantitative score includes:
accessing a clinician-supplied weighting for each of the plurality of component scores; and
applying, to each of the plurality of component scores, the respective clinician-supplied weighting.

11. The method of claim 9, wherein presenting the quantitative score further includes presenting, for each of the component scores, an indication of an amount the respective component score contributes to the quantitative score.

12.-13. (canceled)

14. The method of claim 9, further comprising:
determining that at least one component score of the plurality of component scores is below a respective threshold score;
selecting a preventative care action in response to determining that the at least one component score is below the respective threshold score, wherein the preventative care action is selected to improve the at least one component score; and
facilitating performance of the preventative care action.

15. (canceled)

16. The method of claim 1, wherein the one or more sensors in the environment include (i) at least one radar sensor, (ii) at least one wearable sensor, or (iii) both (i) and (ii).

17. (canceled)

18. The method of claim 1, further comprising:
identifying a change in mental health of the target individual based at least in part on the determined deviation; and
generating a mental health component score based at least in part on the identified change in mental health, wherein generating the quantitative score is based at least in part on the metal health component score.

19. (canceled)

20. The method of claim 1, further comprising generating a physical movement component score based at least in part on the determined deviation, wherein the determined deviation is indicative of i) a change in time to exit a chair; ii) a change in time to sit in a chair; iii) a change in time to cross a room in the environment; iv) a change in time to move from a first point to a second point in the environment; v) a change in gait; or vi) any combination of i-v, wherein generating the quantitative score is based at least in part on the physical movement component score.

21. The method of claim 1, wherein presenting the quantitative score includes presenting one or more changes in score between the quantitative score and one or more past quantitative scores.

22. The method of claim 1, wherein presenting the quantitative score includes:
sorting a plurality of quantitative scores associated with a plurality of individuals in the environment, wherein the target individual is one of the plurality of individuals, and wherein the quantitative score is one of the plurality of quantitative scores; and
presenting the sorted plurality of quantitative scores.

23. The method of claim 1, wherein the routine is indicative of i) a pattern of movement of the target individual through the environment; ii) a pattern of sleep of the target individual within the environment; or iii) a combination of i and ii.

24. The method of claim 1, further comprising:
determining, based at least in part on the quantitative score and the received past sensor data, that a future quantitative score will drop below a threshold value;
selecting a preventative care action based at least in part on the future quantitative score, wherein the preventative care action is selected to improve the future quantitative score; and
facilitating performance of the preventative care action.

25.-28. (canceled)

29. A system comprising:
one or more sensors configured to collect sensor data in an environment;
a control system configured to:
receive, from the one or more sensors, past sensor data, wherein the pas sensor data is associated with a target individual in the environment, and wherein the past sensor data is collected over a plurality of past days;
identify a routine associated with the target individual based at least in part on the past sensor data;
receive, from the one or more sensors, current sensor data, wherein the current sensor data is associated with the target individual in the environment, and wherein the current sensor data is collected after the plurality of past days;
determine a deviation from the routine based at least in part on the identified routine and the received current sensor data;
generate a quantitative score based at least in part on the determined deviation; and
a display device configured to present the quantitative score.

30. The system of claim 29, wherein the control circuit further configured to:
receive (i) healthcare record information associated with the target individual, (ii) past healthcare record information associated with the target individual, or (iii) both (i) and (ii), and wherein (i) the generating the quantitative score is further based at least in part on the received healthcare record information, (ii) the identifying the routine is further based at least in part on the past healthcare record information, or (iii) both (i) and (ii).
